(19) United States
(12) Patent Application Publication
(10) Pub. No.: US 2010/0069262 A1
(43) Pub. Date: Mar. 18, 2010

(54) Method for Cloning Avian-Derived Antibodies

(75) Inventors: Lars Soegaard Nielsen, Nivaa (DK); Charles Pyke, Hilleroed (DK); Anne Marie V. Jensen, Copenhagen OE (DK); Klaus Koefoed, Copenhagen S (DK); Allan Jensen, Fredensborg (DK); Mette Thorn, Copenhagen S (DK)

Correspondence Address: STERNE, KESSLER, GOLDSTEIN & FOX P.L.L.C., 1100 NEW YORK AVENUE, N.W., WASHINGTON, DC, 20005, US

(73) Assignee: Symphogen A/S, Lyngby (DK)

(21) Appl. No.: 12/549,921

(22) Filed: Aug. 28, 2009

Related U.S. Application Data
(60) Provisional application No. 61/136,390, filed on Sep. 2, 2008.

(30) Foreign Application Priority Data
Aug. 29, 2008 (DK) PA 2008 01190

(51) Int. Cl. C40B 40/08; C40B 50/06; C12P 19/34; C12N 15/74; C12P 21/02; C12M 1/00

(52) U.S. Cl. 506/17; 506/26; 435/91.2; 435/320.1; 435/69.1; 435/289.1

(57) ABSTRACT

The invention relates to a procedure for linking cognate pairs of VH and VL encoding sequences from a population of avian cells enriched in particular surface antigen markers. The linking procedure involves a multiplex molecular amplification procedure capable of linking nucleotide sequences of interest in connection with the amplification (multiplex PCR). The method is particularly advantageous for the generation of cognate pair libraries as well as combinatorial libraries of antibody variable region encoding sequences from chickens or other birds. The invention also provides methods for generation of chimeric human/avian antibodies and expression libraries generated by such methods.

### REFERENCE TO A SEQUENCE LISTING SUBMITTED ELECTRONICALLY VIA EFS-WEB

The content of the electronically submitted sequence listing (Name: P123PC_sequencelist_ST25.txt; Size 16 kilobytes; and Date of Creation: Aug. 27, 2009) filed herewith the application is incorporated by reference in its entirety).

### FIELD OF THE INVENTION

The present invention relates to a method for linking cognate pairs of antibody heavy chain and light chain encoding sequences from a population of avian-derived cells enriched in particular surface antigen markers. The method involves a multiplex molecular amplification procedure capable of linking nucleotide sequences of interest in connection with the amplification, in particular polymerase chain reaction (multiplex PCR). The method is particularly advantageous for the generation of cognate pair libraries as well as combinatorial libraries of variable region encoding sequences from immunoglobulins. The invention also relates to methods for generation of chimeric human/avian antibodies and expression libraries generated by such methods.

### BACKGROUND OF THE INVENTION

WO 2005/042774 discloses a method for linking nucleotide sequences of interest, in particular cognate pairs of antibody heavy chain variable region and light chain variable region (V_{H} and V_{L}) encoding sequences, using a multiplex molecular procedure. The sequences of interest are preferably amplified and linked from isolated single cells following limiting dilution or other cell separation techniques. This document discloses various ways of enriching a lymphocyte-containing cell population to obtain a population of antibody producing/encoding cells, e.g. plasma cells, that are particularly suitable for the multiplex molecular amplification procedure.

WO 2008/104184 discloses methods for generating libraries of immunoglobulin encoding sequences from a mouse or other rodent using a multiplex amplification procedure performed on a population of isolated single cells enriched with the surface antigens CD43 and CD138 or MHCII and B220.

The methods described in these documents, generally referred to as the Symplex™ technology, are especially adapted to generation of libraries of cognate pairs of variable region encoding sequences derived from human cells or from the cells of a mouse or other rodent. However, these documents do not address the issue of generating libraries of cognate pairs or combinatorial libraries from the cells of a chicken or other bird. This approach, for the purpose of generating avian antibodies or, preferably, chimeric human/avian antibodies, is of interest given the phylogenetic differences between humans on the one hand and chickens or other birds on the other hand. This is due to the fact that therapeutic antibodies for treatment of various human diseases and conditions are often generated from mice, but since mice and humans are, relatively speaking, closely related species, there may be human diseases and conditions where optimal antibodies against human antigens may not be able to be generated in mice or other mammalian species. This may in particular be the case for antibodies targeting human self-antigens aimed at treating cancer or autoimmune diseases. In such cases, it can be advantageous to be able to isolate antibodies of interest from a species that is phylogenetically distal to humans such as a to chicken or other bird. The present invention addresses the problem of how to isolate avian cells that produce antibodies of potential interest for use as human therapeutics, so as to be able to ultimately identify new and useful antibody treatments.

### SUMMARY OF THE INVENTION

The present invention focuses on methods for generating libraries of immunoglobulin encoding sequences from birds and methods for generating libraries of vectors coding for chimeric antibodies comprising human constant regions and avian variable regions. The methods of the invention involve relatively few steps and are adapted for high throughput screening and cloning.

In a first aspect the invention relates to a method for producing a library of cognate pairs comprising linked variable region encoding sequences, said method comprising:
a) providing a lymphocyte-comprising cell fraction from a donor of avian origin;
b) obtaining a population of isolated single cells by distributing cells from said cell fraction individually into a plurality of vessels, wherein at least a subpopulation of the cells expresses immunoglobulin genes, preferably IgY, and optionally any avian B cell marker antigen; and
c) amplifying and effecting linkage of the variable region encoding sequences contained in said population of isolated single cells by amplifying, in a multiplex molecular amplification procedure, nucleotide sequences of interest using a template derived from an isolated single cell or a population of isogenic cells, and effecting linkage of the amplified nucleotide sequences of interest.

This method provides a library of cognate pair antibodies or antibody fragments.

In another aspect the invention relates to a method of randomly linking a plurality of non-contiguous nucleotide sequences of interest, comprising:
a) amplifying, in a multiplex molecular amplification procedure, nucleotide sequences of interest using a template derived from a population of genetically diverse cells, wherein the genetically diverse cells are derived from a lymphocyte-comprising cell fraction of avian origin, and wherein at least a subpopulation of the cells expresses immunoglobulin genes, preferably IgY, and optionally any avian B cell marker antigen; and
b) effecting linkage of the nucleotide sequences of interest amplified in step a).

This method provides a combinatorial library of randomly combined heavy and light chain variable region encoding domains.

The immunoglobulin-expressing subpopulation of cells of the present invention may in particular be characterized by, and may be assessed for and/or enriched for, any of the following:
expression of IgY (IgY⁺),
expression of IgY, and CD3 negative (IgY⁺CD3⁻),
expression of IgY, no or low expression of Bu-1, and CD3 negative (IgY⁺Bu-1⁻CD3⁻),
expression of Bu-1 and IgY (Bu-1⁺IgY⁺),
expression of Bu-1 and IgY, and CD3 negative (Bu-1⁺IgY⁺CD3⁻),
expression of Bu-1 but not any monocyte markers (Bu-1⁺, monocyte⁻),
expression of Bu-1 and no or low levels of IgM (Bu-1⁺IgM⁻), or
expression of Bu-1 and BAFF (Bu-1⁺BAFF⁺).

The subpopulation of cells is in particular characterized by expression of the avian immunoglobulin IgY. This subpopulation may be further characterized by expression and/or lack of expression of any one or more avian B cell marker antigens, as it may be expected that the subpopulation of cells that expresses immunoglobulin genes such as IgY will also express a detectable level of at least one avian B cell marker antigen. The subpopulation may thus defined in terms of expression, optionally a particular level of expression, of a detectable level of any one or more avian B cell marker antigens, and/or in terms of lack of expression of any one or more avian B cell marker antigens, where the avian B cell marker antigens may e.g. include one or more of Bu-1, CD3, IgM or BAFF. In a particular embodiment, the subpopulation is characterized by expression of IgY and by being CD3 negative (CD3⁻; i.e. no or negligible expression of CD3). In a further particular embodiment, the subpopulation is characterized by expression of IgY, by no or low expression of Bu-1, and by being CD3 negative.

Other marker antigens of interest may also include avian orthologs of human B cell markers such as CD19, CD20, CD27, CD38 or CD45; or avian orthologs of murine B cell markers such as MHCII, B220, CD43, or CD138; or a combination of these markers. Experimental data provided in the present application establish that cell populations isolated from chicken-derived splenocytes based on expression of IgY, optionally combined with sorting for expression or lack of expression of surface antigens such as Bu-1 and/or CD3, provide a good starting material for cloning of antibody-encoding sequences using a multiplex molecular amplification method. The methods of the invention can easily be applied to other species expressing orthologs of IgY and optionally other avian B cell marker antigens. The methods can in particular be applied to other avian species, for example a duck, goose, pigeon or turkey.

Furthermore, the method of the invention provides a library of polynucleotides that can be easily sequenced and/or inserted into vectors, such as expression, transfer, display or shuttle vectors, so that once a particular antibody has been selected, it is cloned, its sequence is determined and it can be easily transferred to an appropriate expression vector for production of a recombinant antibody.

It is expected that cells sorted according to the protocol disclosed herein will be capable of providing a source of high affinity antibodies, potentially with affinities in the picomolar range. Monoclonal antibodies from hybridomas may not possess affinities in the picomolar range, and will then need to be synthetically affinity matured to reach such affinities.

In one embodiment, these methods further comprise assessing, prior to multiplex molecular amplification, that the population of lymphocyte-comprising cells comprises cells defined by expression of avian immunoglobulin genes, in particular IgY, and optionally by expression (presence or absence, or a particular level of expression) of one or more avian B cell surface markers according to the criteria discussed above, preferably CD3 and/or Bu-1, e.g. that the population comprises cells expressing detectable levels of IgY and/or Bu-1. Also, the methods may include enriching said lymphocyte-comprising cell fraction for a lymphocyte population defined in terms of expression of IgY and expression and/or lack of expression of one or a combination of bird B cell surface markers, preferably CD3 and/or Bu-1 as discussed above, e.g. enriching for cells that express IgY and that are characterized by expression or lack of expression of e.g. Bu-1 and/or CD3, prior to multiplex molecular amplification. In a particular embodiment, the population is assessed for, and/or enriched for, cells that express IgY. In other particular embodiments, the population may be assessed for, and/or enriched for, cells that express IgY and that are CD3 negative, or that express IgY and Bu-1, or that express IgY and that express no or low levels of Bu-1.

Preferably, the methods further comprise isolating from said lymphocyte-comprising population single cells expressing immunoglobulin genes and an avian B cell antigen prior to the multiplex molecular amplification. In a preferred embodiment, the isolated single cells or subpopulation of cells are characterized by their expression profiles of IgY, Bu-1 and/or CD3 as being positive or negative, or high, intermediate or low relative to the lymphocyte-comprising cell fraction, i.e. in accordance with the criteria discussed above. In a preferred embodiment the isolated single cells of the subpopulation of cells are IgY⁺ and/or Bu-1⁺, preferably IgY⁺, for example CD3⁻/Bu-1⁻/IgY⁺. Enrichment or isolation preferably comprises an automated sorting procedure, such as flow cytometry, in particular fluorescence activated cell sorting (FACS). Alternatively, sorting may be performed using magnetic bead cell sorting (MACS)

In a further aspect, the invention relates to a method for generating a vector encoding a chimeric antibody with human constant regions and non-human variable regions, said method comprising:
a) providing a lymphocyte-comprising cell fraction from a donor of avian origin;
b) obtaining a population of isolated single cells by distributing cells from said cell fraction individually into a plurality of vessels;
c) amplifying and effecting linkage of the variable region encoding nucleic acids contained in said population of isolated single cells by amplifying, in a multiplex molecular amplification procedure, said nucleic acids using a template derived from an isolated single cell or a population of isogenic cells; and effecting linkage of the amplified nucleic acids encoding variable regions of heavy and light chains;
d) effecting linkage of the amplified variable regions to human constant regions; and
e) inserting the obtained nucleic acid into a vector.

Preferably, the bird species is a chicken. To the extent that the methods of the invention are applied to chicken/hen derived cells, the methods are named: chicken Symplex™ or chSymplex™.

By this aspect of the invention there is provided a novel method for generation of libraries of chimeric human/avian antibodies. This is made possible by combining the multiplex molecular amplification and subsequent cloning into a vector backbone with ligation and/or splicing of human heavy and light chain constant domains. Traditionally, in a method for generating chimeric human/avian antibodies, the chimerisation has been the last step after hybridomas have been established and screened and the encoded antibody has been cloned. Chimerisation may affect the binding specificity and/or affinity of an antibody, and thus there is a risk that a good monoclonal chicken antibody will lose its efficacy when it is chimerised into a human/chicken antibody.

By the provision of a method that directly generates an antibody repertoire of chimeric antibodies, the screening can be carried out on products that may not need to be modified further prior to preclinical and clinical development.

The constant human regions can be provided in a molecular amplification step or they can be provided as part of a vector backbone into which the variable regions are cloned following molecular amplification. In a preferred embodiment the method comprises a further amplification step, wherein a polynucleotide encoding a human constant light chain, or a fragment thereof with an overlap capable of providing linkage to the variable light chain, is added to the PCR mixture together with a primer set capable of amplification of a construct comprising, in order: a chicken VH chain, a linker, a chicken VL chain, and a human constant light chain.

In another embodiment, the method comprises a further amplification step, wherein a polynucleotide encoding a human constant heavy chain, or a fragment thereof with an overlap capable of providing linkage to the variable heavy chain, is added to the PCR mixture together with a primer set capable of amplification of a construct comprising, in order: a human constant heavy chain, a chicken VH chain, a linker, and a chicken VL chain.

Consequently, there is also provided a library of vectors encoding chimeric antibodies, each antibody member consisting of avian immunoglobulin variable region encoding sequences and human immunoglobulin heavy and light chain constant regions.

Preferably, the vectors are expression vectors enabling the expression of the antibody members of the library for subsequent screening for antigen specificity. More preferably, the expression vector is for mammalian expression. The vectors of the library may be obtained by a method of the invention.

In one embodiment, the light chain constant region is a human lambda or kappa constant region.

The avian sequences may be from a donor of any avian origin for which sequence information is available to allow the design of suitable primers, and for which suitable cell sorting techniques enable sorting of antibody producing or encoding cells for single cell multiplex molecular amplification to link cognate pairs of variable region sequences.

Preferably, the variable regions of the antibodies are cognate pairs.

In another aspect, the invention relates to a sub-library which codes for antibodies exhibiting desired binding specificities directed against a particular target, selected from a library according to the invention.

In a further aspect the invention provides a multiwell plate comprising, in the majority of wells, one cell derived from a lymphocyte-comprising cell fraction from an avian donor, said cell expressing immunoglobulin genes including IgY and/or Bu-1 antigen, and buffers and reagents required for carrying out reverse transcription of mRNA and for amplifying variable heavy and light chain encoding regions.

In a further aspect the invention provide a method for producing a library of avian-derived immunoglobulin variable region encoding sequences, said method comprising:
a) providing a lymphocyte-comprising cell fraction from a donor of avian origin;
b) obtaining a population of isolated single cells by distributing cells from said cell fraction individually into a plurality of vessels, wherein at least a subpopulation of the cells express immunoglobulin genes, e.g. IgY, and optionally at least one avian B cell marker antigen; and
c) amplifying the variable region encoding sequences contained in said population of isolated single cells by amplifying, in a multiplex molecular amplification procedure, nucleotide sequences of interest using a template derived from an isolated single cell or a population of isogenic cells.

By this method, a library of avian-derived immunoglobulin variable region encoding sequences may be obtained from a subpopulation of cells that expresses avian immunoglobulin genes as generally described herein. The method may comprise a further step of effecting linkage of heavy chain and light chain variable region encoding sequences so as to obtain a library of cognate pairs, i.e. a library of avian-derived antibodies or fragments thereof.

### DESCRIPTION OF THE FIGURES

FIG. 1: Principles of multiplex RT-PCR and nested amplification of chicken variable regions. The following primer abbreviations are used: CH-HCrev: chicken IgY heavy chain constant region antisense primer, CH-VH: chicken heavy chain variable region 5′ sense primer, CH-VL: chicken light chain variable region 5′ sense primer, CH-LCrev: chicken light chain constant region antisense primer, CH-JH: chicken heavy chain J-region antisense primer, CH-JL: chicken light chain J-region antisense primer.

FIG. 2: Principles of addition of human heavy chain and light chain constant regions by overlap extension PCR, cloning into vector backbone and addition of mammalian promoter-leader fragment. Human heavy and light chain constant regions are amplified with overlap for the appropriate J-region. The following primer abbreviations are used: hCHC-R: human IgG1 constant region 3′ antisense primer, hL-R: human lambda constant region 3′ antisense primer.

FIGS. 3 to 9 show dot plots of chicken splenocytes stained for various surface markers (Bu-1, CD3, IgY, and IgY specific for TT). The chickens were immunized with tetanus toxoid and the spleens were harvested at day 10 after the 3^{rd} boost with TT in incomplete Freund's adjuvant (IFA). See Example 1.

FIG. 3: Within this population of splenocytes, 3 gates were set: (1) Bu-1⁺CD3⁻ cells (upper left), (2) an intermediary population, P2 (Bu-1^{low}CD3^{-/low}), (3) Bu-1⁻CD3⁻ cells (lower left).

FIG. 4: Among Bu-1⁺CD3⁻ cells, an additional IgY gate was included.

FIG. 5: Among Bu-1⁺CD3⁻IgY⁺ cells, the TT⁺ cells were gated.

FIG. 6: Among Bu-1⁻CD3⁻ cells, an additional IgY⁺ gate was included.

FIG. 7: Among Bu-1⁻CD3⁻IgY⁺ cells, the TT⁺ cells were gated.

FIG. 8: Among the intermediary population, P2, a new gate was defined as IgY⁺ cells (P3).

FIG. 9: Among Bu-1^{low}CD3^{-/low}IgY⁺ cells (P3), the TT⁺ cells were gated.

FIG. 10 shows an agarose gel containing 21 Symplex reaction products with the expected electrophoretic mobility from Example 4, in which a chimeric chicken-human anti-tetanus toxoid antibody repertoire was cloned. Size markers (500, 1000, 1500 etc. base pair bands) are also shown.

FIG. 11 shows the reaction products (the approximately 2 kb overlap band) after overlap extension PCR performed on a mixture of purified chicken VH-VL, human lambda light chain constant region and human IgG1 heavy chain constant region encoding sequences.

FIG. 12 shows an alignment of VH regions from 10 randomly chosen clones from antibodies isolated from spleen B cells from a chicken immunized with tetanus toxoid by Symplex PCR. The CDR3 regions with short stretches of flanking framework and human HC constant region are shown. The sequences shown are, from top to bottom, subsequences of SEQ ID NO:13 to SEQ ID NO:22.

FIGS. 13 and 14 show alignments of CDR3 regions of VH (FIG. 13) and VL (FIG. 14) of five tetanus toxoid-specific clones.

FIG. 13: VH CDR3 alignment; the sequences are, from top to bottom, subsequences of SEQ ID NO:23 to SEQ ID NO:27.

FIG. 14: VL CDR3 alignment; the sequences are, from top to bottom, subsequences of SEQ ID NO:28 to SEQ ID NO:32.

### DETAILED DESCRIPTION OF THE INVENTION

The present invention provides further possibilities for using the general amplification and linkage method disclosed in WO 2005/042774 for providing collections of antibody vectors from birds. These improvements enable the cloning of human/avian chimeric antibody encoding sequences with cognate pairs of variable regions suitable for use in a high-throughput format. This is achieved by providing a new starting material for the amplification and linkage processes and by providing methods for generation of libraries of chimeric human/avian antibodies with cognate pairs of variable regions.

One aspect of the invention is a method of linking heavy and light chain variable sequences by amplifying, in a multiplex molecular amplification procedure, the relevant avian nucleotide sequences using a template derived from an isolated single cell, a population of isogenic cells or a population of genetically diverse cells, and effecting a subsequent linkage of the amplified sequences.

### DEFINITIONS

The term “cognate pair” describes an original pair of non-contiguous nucleic acids of interest that are contained within or derived from a single cell. In preferred embodiments, a cognate pair comprises two variable region encoding sequences which together encode a binding protein variable domain and which are derived from the same cell. Thus, when expressed either as a complete binding protein or as a stable fragment thereof, they preserve the binding affinity and specificity of the binding protein originally expressed from this cell. A cognate pair can for example be an antibody variable heavy chain encoding sequence associated with a variable light chain encoding sequence from the same cell, or a T cell receptor α chain encoding sequence associated with a β chain encoding sequence from the same cell. A library of cognate pairs is a collection of such cognate pairs.

The term “isogenic population of cells” describes a population of genetically identical cells. In particular, an isogenic population of cells derived by clonal expansion of an isolated single cell is of interest in the present invention.

The term “isolated single cell” describes a cell that has been physically separated from a population of cells, corresponding to “a single cell in a single vessel”. When distributing a population of cells individually among a plurality of vessels, a population of isolated single cells is obtained. As specified in the section entitled “Template sources”, the proportion of vessels with a single cell does not necessarily have to be 100% in order for it to be considered a population of single cells.

The terms “high”, “intermediate” and “low” in the context of expression levels of an antigen marker on the surface of cells are relative measures based on the relative fluorescence intensity of a subset of cells compared to the complete population of analyzed cells in any given analysis or sorting procedure. A “negative” population of cells is often defined by a fluorescence intensity of below about 10³ mean fluorescence units. The fraction of a cell population designated as “low” may be similar to the negative population, but may also be just below the “intermediate” cell population, where an intermediate population has a fluorescence intensity higher than the low cell population but less that the cell fraction giving the highest fluorescence intensity, which is often above about 10⁴ mean fluorescence units. It should be emphasized that the definitions of high, intermediate, low or negative are relative to the individual analysis, and that the mean fluorescence unit values cited here are typical values that are exemplary but not necessarily limiting. This will be understood by persons skilled in the art of flow cytometry techniques such as FACS, who will be readily able to characterize the results of any particular flow cytometry procedure.

The terms “link” or “linkage” in relation to amplification describe the association of the amplified nucleic acid sequences encoding the nucleic acid sequences of interest into a single segment. In relation to cognate pairs, a single segment comprises nucleic acid sequences encoding a variable domain, e.g. an antibody heavy chain variable region associated with an antibody light chain variable region encoding sequence, where the two variable region encoding sequences are derived from the same cell. The linkage can either be achieved simultaneously with the amplification or as a separate step following the amplification. There are no requirements as to the form or functionality of the segment; it may be linear, circular, single-stranded or double-stranded. Nor is the linkage necessarily permanent, as one of the nucleic acid sequences of interest may be isolated from the segment if desired. One of the variable region encoding sequences may for example be isolated from a cognate pair segment. However, as long as the original variable regions constituting the cognate pair are not scrambled with other variable regions, they are still considered a cognate pair, even though they may not be linked together into a single segment. The linkage is preferably a nucleotide phosphodiester linkage. However, linkage can also be obtained by different chemical cross linking procedures.

The term “multiplex molecular amplification” describes the simultaneous amplification of two or more target sequences in the same reaction. Suitable amplification methods include the polymerase chain reaction (PCR), ligase chain reaction (LCR), (Wu and Wallace, 1989, Genomics 4, 560-9), the strand displacement amplification (SDA) technique (Walker et al., 1992, Nucl. Acids Res. 20, 1691-6), self-sustained sequence replication (Guatelli et al., 1990, Proc. Nat. Acad. Sci. USA, 87, 1874-8) and nucleic acid based sequence amplification (NASBA) (Compton J., 1991, Nature 350, 91-2). The latter two amplification methods involve isothermal reactions based on isothermal transcription, which produce both single stranded RNA (ssRNA) and double stranded DNA (dsDNA).

The term “multiplex PCR” describes a variant of PCR in which two or more target sequences are amplified simultaneously, by including more than one set of primers in the same reaction, e.g. one primer set adapted for amplification of the heavy chain variable region and one primer set adapted for amplification of the avian light chain variable region in the same PCR reaction.

The term “multiplex RT-PCR” describes a multiplex PCR reaction which is preceded by a reverse transcription (RT) step. The multiplex RT-PCR can either be performed as a two-step process with a separate RT step prior to the multiplex PCR, or as a single-step process where all components for both RT and multiplex PCR are combined in a single vessel.

The terms “multiplex overlap-extension PCR” and “multiplex overlap-extension RT-PCR” imply that the multiplex PCR or multiplex RT-PCR is performed utilizing a multiplex overlap-extension primer mix to amplify the target sequences, thereby enabling simultaneous amplification and linkage of the target sequences.

The term “a plurality of vessels” describes any object (or collection of objects) which enables the physical separation of a single cell from a population of cells. This may be tubes, multiwell plates (e.g. 96-well, 384-well, microtiter plates or other multiwell plates), arrays, microarrays, microchips, gels, or a gel matrix. Preferably the object is applicable for PCR amplification. The terms “tubes” or “vessels” may be used interchangeably herein.

The term “polyclonal protein” or “polyclonality” as used herein refers to a protein composition comprising different, but homologous protein molecules, preferably selected from the immunoglobulin superfamily. Thus, each protein molecule is homologous to the other molecules of the composition, but also contains at least one variable polypeptide subsequence characterized by differences in the amino acid sequence between the individual members of the polyclonal protein. Known examples of such polyclonal proteins include antibody or immunoglobulin molecules, T cell receptors and B cell receptors. A polyclonal protein may consist of a defined subset of protein molecules defined by a common feature such as shared binding activity towards a desired target, e.g. a polyclonal antibody exhibiting binding specificity towards a desired target antigen.

The terms “immunoglobulin” and “antibody” may be used interchangeably herein.

The term “a population of genetically diverse cells” as used herein refers to a cell population in which the individual cells differ from each other at the genomic level. Such a population of genetically diverse cells may for example be a population of cells derived from a donor, or a fraction of such cells, e.g. a B lymphocyte or a T lymphocyte containing cell fraction.

The term “primer pair” describes two primers capable of priming the amplification of a nucleotide region of interest, whereas the term “primer set” describes two or more primers which together are capable of priming the amplification of a nucleotide sequence of interest. A primer set thus includes at least one primer pair, but may include more than two primers and will often include multiple primer pairs. A primer set of the present invention may be designed to prime a family of nucleotide sequences containing variable region encoding sequences. Examples of different families are antibody kappa light chains, lambda light chains and heavy chain variable regions. A primer set for the amplification of a family of nucleotide sequences containing variable region encoding sequences often constitutes a plurality of primers in which several primers can be degenerate primers.

The term “sequence identity” is expressed as a percentage which indicates the degree of identity between two nucleic acid sequences over the length of the shortest of the two sequences. It can be calculated as (Nref−Ndif)×100%/Nref, wherein Nref is the number of residues in the shorter of the sequences, and wherein Ndif is the total number of non-identical residues in an Nref long optimally aligned match between the two sequences. For example, the DNA sequence AGTCAGTC will have a sequence identity of 75% with the sequence TAATCAATC (Ndif=2 and Nref=8) (underlining shows the optimal alignment, and bold indicates the 2 non-identical residues out of 8).

The terms “randomly” or “random” with respect to linkage refer to linkage of nucleotide sequences which are not derived from the same cell. If the nucleotide sequences of interest are variable region encoding sequences, this will result in a combinatorial library of linked sequences. If, on the other hand, the nucleotide sequences of interest encode a non-diverse heteromeric protein, the randomly linked sequences will appear similar to sequences linked from a single cell.

The term “template derived from an isolated single cell,” in the context of reverse transcription, relates to the nucleic acids within such an isolated cell. The nucleic acids can for example be in the form of mRNA or other RNA, or genomic or other DNA. The nucleic acids can either be isolated from the cell or be associated with other contents of the cell, where the cell is intact or lysed.

The term “Bu-1” refers to a specific chicken surface antigen, also known under synonyms including chB6 and Bu1. Two different highly homologous chicken Bu-1 proteins are known. These are referred to as Bu-1a (Uniprot accession No. Q90746) and Bu-1b (Uniprot accession No. Q90747). Both have a length of 335 amino acid residues, and the sequence of the two is identical other than in a very few residues. As used herein, the term “Bu-1” is intended to cover both Bu-1a and Bu-1b.

The term “IgY” refers to the main serum immunoglobulin in chickens, also known under the synonym chicken IgG.

The term “BAFF” refers to B cell activating factor, also known under the synonyms BlyS, TALL-1, THANK and zTNF4.

The terms “avian” and “bird” may be used interchangeably herein and are intended to include, for example, chickens, ducks, geese, pigeons and turkeys. A preferred bird for use in the present invention is a chicken.

The term “chicken” as used herein refers generally to members of the species Gallus gallus, in particular domesticated chickens of the subspecies Gallus gallus domesticus, and is intended to include both hens and roosters/cocks, i.e. both females and males.

The letters “ch”, when used in terms such as “chVH”, refer to chicken-derived sequences.

The term “ortholog” as used herein refers to a gene in two or more species that has evolved from a common ancestor. An avian gene encoding an ortholog of, e.g., a human B cell marker will generally encode a protein that has the same or a similar function as the protein encoded by the orthologous human gene.

The term “hot-start polymerase” describes polymerases that are inactive or have very low activity at temperatures used for reverse transcription. Such polymerases need to be activated by high temperatures (90 to 95° C.) to become functional. This is for example an advantage in single-step RT-PCR procedures, since it prohibits interference of the polymerase with the reverse transcriptase reaction.

### Sequences of Interest

The nucleotide sequences of interest that may be linked according to the present invention can be selected from sequences that encode different subunits or domains whose expression products are a protein or part of a protein. In particular, the encoded proteins or parts thereof are heteromeric proteins, i.e. proteins that are composed of at least two non-identical subunits. Some of the classes to which such proteins belong are for example enzymes, inhibitors, structural proteins, toxins, channel proteins, G-proteins, receptor proteins, immunoglobulin superfamily proteins, transportation proteins etc. The nucleotide sequences encoding such heteromeric proteins are non-contiguous, meaning that they e.g. originate from different genes, or different mRNA molecules. However, non-contiguous as used in the context of the present invention may also mean nucleotide sequences encoding domains of the same protein, where the domains are separated by nucleotide sequences which are not of interest.

In one embodiment of the present invention the nucleotide sequences of interest contain variable region encoding sequences from the immunoglobulin superfamily, such as immunoglobulins (antibodies) or B cell receptors. Variable region encoding sequences from immunoglobulins are of particular interest. Such variable region encoding sequences comprise full-length antibodies as well as fragments thereof such as Fabs, Fvs, scFvs, or combinations of fragments of the variable region encoding sequences, e.g. complementarity determining regions (CDRs), joining genes or V-genes or combinations of these. Generally, the present invention can be applied to any combination of variable region encoding sequences and fragments thereof. For example, the invention enables the linkage of variable domains of antibody heavy and light chains, thereby generating Fv or scFv encoding sequences, or alternatively e.g. linkage of the entire light chain with the heavy chain variable region+constant region domain C_{H1}+parts of the hinge region, generating Fab, Fab′ or F(ab)₂. Further, it is possible to add any region of the heavy chain constant region domains to the variable heavy chain, thereby generating full-length antibody encoding sequences or truncated antibody encoding sequences. In one aspect of the invention, non-human, avian-derived variable sequences are linked to human constant regions to generate chimeric human/avian antibodies.

### Template Sources

The invention allows linkage of nucleotide sequences derived from an isolated single cell (where each individual cell is located in a single well or other vessel), a population of isogenic cells, or a genetically diverse population of cells which have not been separated into single vessels.

A preferred feature of the present invention is the use of isolated single cells or a population of isogenic cells as the template source, since scrambling of the nucleic acid sequences of interest, i.e. linkage of sequences derived from different cells, is avoided. This is of particular importance in the case of antibody variable region encoding sequences, where the aim is to obtain a cognate pair of variable region encoding sequences or CDR encoding sequences.

Preferably, the invention is performed on a single cell or a population of single cells from a cell fraction comprising lymphocytes, such as B lymphocytes, plasma cells and/or various developmental stages of these cell lineages. Other populations of cells that express binding proteins from the immunoglobulin superfamily may also be used to obtain single cells. Cell lines such as hybridoma cells, cell lines of B lymphocyte lineage, or virus immortalized cell lines or donor derived cells participating in the immune response may also be used in the present invention. Donor derived lymphocyte-containing cell fractions may be obtained from natural tissue or fluid that is rich in such cells, e.g. blood, bone marrow, lymph nodes, spleen tissue, tonsil tissue, bursa fabricii, or from infiltrations in and around tumors or inflammatory tissue infiltrations. Preferably, spleen tissue, blood, bursa fabricii or bone marrow is used. Donors can either be naïve or hyperimmune birds with respect to a desired target. In a particularly preferred embodiment, the donor is a chicken or other bird that has been immunised with a human self-antigen, such as a human protein implicated in cancer or inflammatory diseases, for example EGFR or TNFα.

The donor may also be a transgenic bird, preferably a transgenic chicken carrying human immunoglobulin sequences capable of producing immunoglobulins derived from or having significant similarity to human antibody variable heavy and light chains. Human antibodies against a specific target can be raised by immunization of such transgenic birds with a desired antigen using standard immunization techniques. This allows for generation of libraries encoding antibodies directed against targets that are difficult or impossible to generate antibodies against using e.g. mice or other animals that are more closely related to humans. This approach is envisaged to be particularly useful in cases such as human antigens for which no natural human antibody response or only a limited response exists.

The use of chickens, in particular transgenic chickens, or other birds as donors is expected to be advantageous in that they can provide an alternative humoral response compared to antibody responses from mice and other mammals. The distal phylogenetic relationship between chickens/birds and humans allows antibody responses against epitopes that in species more closely related to humans than birds, e.g. mice, rats or non-human primates, would not be immunogenic due to sequence homology. The extended diversity that is contemplated to be obtainable by the chicken antibody response has the potential to increase the frequency of identified antibodies with therapeutically potential and furthermore to allow identification of antibodies that are cross-reactive between human and mice antigen orthologs, which may facilitate preclinical studies in mice disease models.

In one embodiment, the lymphocyte-containing cell fraction comprises whole blood, bone marrow, mononuclear cells or white blood cells obtained from a donor. Mononuclear cells can be isolated from blood, bone marrow, lymph nodes, spleen, bursa fabricii, infiltrations around cancer cells or inflammatory infiltrations. Mononuclear cells can be isolated by density centrifugation techniques, e.g. Ficoll gradients. If the mononuclear cells are isolated from samples composed of tissue, the tissue is disintegrated before the gradient centrifugation is performed. Disintegration can be performed e.g. by mechanical methods such as grinding, electroporation and/or by chemical methods such as enzymatic treatment. Raw preparations of for example bone marrow or tissue that contains lymphocytes can also be used. Such preparations will need to be disintegrated, for example as described above, in order to facilitate single cell distribution.

In a preferred embodiment, the lymphocyte-containing cell fraction, e.g. whole blood, mononuclear cells, white blood cells or bone marrow, is enriched with respect to a particular lymphocyte population, such as cells from the B lymphocyte lineage. Enrichment of B lymphocytes can for example be performed using magnetic bead cell sorting (MACS) or fluorescence activated cell sorting (FACS), taking advantage of lineage-specific cell surface marker proteins such as Bu-1 or other avian B cell lineage-specific markers such as IgY. Alternatively, chicken orthologs of known human or murine B cell markers may be used.

A preferred feature of the present invention is to sort the enriched B lymphocytes further in order to acquire plasma cells, before distributing the cells individually among a plurality of vessels. Isolation of plasma cells is generally performed by MACS sorting or FACS sorting, utilizing the expression profile of surface markers such as IgY, CD3, Bu-1, IgM, monocyte markers and BAFF. As described above, sorting and selection of cells is based on the absence or presence of expression of one or more of these markers, e.g. defining the expression as being low, medium or high relative to the lymphocyte-comprising cell population from which they are selected or isolated. Other plasma cell-specific surface markers or combinations thereof can be utilized as well, for example the chicken orthologs of CD138, CD43, CD19 or MHC-II. The exact choice of marker depends on the plasma cell source, e.g. spleen, bursa fabricii, tonsils, blood or bone marrow, as well as the species from which the cells are isolated.

As described above, a preferred marker for use in the present invention is IgY, and in a preferred embodiment the cells that are selected are IgY⁺. In further particular embodiments based on IgY cells, the cells that are selected may be IgY⁺, CD3⁻; or they may be IgY⁺, Bu-1⁻, CD3⁻. Alternatively or additionally, cells may be selected based in part on expression (or lack of expression) of Bu-1. Particular embodiments based on Bu-1 expression are selection of cells that are Bu-1⁺IgY⁺; Bu-1⁺IgY⁺CD3⁻; Bu-1⁺monocyte⁻; Bu-1⁺IgM⁻; or Bu-1⁺BAFF⁺.

Plasma cells can also be obtained from a non-enriched lymphocyte-containing cell population obtained from any of these sources. The plasma cells isolated from blood are sometimes called early plasma cells or plasma blasts. In the context of the present invention these cells are also considered to be “plasma cells”. Plasma cells are desired for the isolation of cognate pairs of immunoglobulin encoding sequences because, compared to other B lymphocyte cells, a higher frequency of these cells produces antigen-specific antibodies that reflect the acquired immunity toward the desired antigen, and most of the cells have undergone somatic hypermutation and therefore encode high-affinity antibodies. Further, the mRNA levels in plasma cells are elevated compared to the remaining B lymphocyte population, so that the reverse transcription procedure is more efficient when using single plasma cells. As an alternative to plasma cell isolation, memory B cells may be isolated from a lymphocyte containing cell fraction utilizing a cell surface marker such as IgY or the expression level of the chicken ortholog of the human CD27 B cell surface marker.

In one embodiment, the enriched B lymphocytes may be selected for antigen specificity before distributing the cells among a plurality of vessels. Isolation of antigen-specific B lymphocytes is performed by contacting the enriched B lymphocytes with the desired antigen or antigens, enabling binding of antigen to surface-exposed immunoglobulin, followed by isolation of binders. This can be done, for example, by biotinylation of the desired antigen or antigens followed by suitable cell sorting techniques. Plasma cells as well as B lymphocytes, non-enriched mononuclear cells, white blood cells, whole blood, bone marrow or tissue preparations can be subjected to isolation with respect to antigen specificity if so desired.

As an alternative to sorting cells expressing certain surface markers, i.e. a positive selection, it is conceivable that cells not expressing certain markers are depleted from the composition of cells, leaving the cells behind that actually express the markers.

If desired, any of the isolated cell fractions described above (e.g. B lymphocytes, plasma cells, memory cells) may be immortalized. Immortalization may for example be performed prior to cell distribution. Alternatively, isolated single cells may be immortalized and expanded prior to reverse transcription.

In one embodiment, a population of desired cells (e.g. hybridoma cells, cell lines of B lymphocyte lineage, whole blood cells, bone marrow cells, mononuclear cells, white blood cells, B lymphocytes, plasma cells, antigen-specific B lymphocytes, memory B cells) are distributed individually into a plurality of vessels in order to obtain a population of isolated single cells. This isolation of single cells refers to the physical separation of cells from a population of cells in such a way that a single vessel contains a single cell, or a micro array, chip or gel matrix is loaded in a manner that results in isolated single cells. The cells may be distributed directly into a multitude of vessels such as arrays of single vessels by limiting dilution. The single vessels utilized in the present invention are preferably those suitable for PCR (e.g. PCR tubes and 96 well or 384 well PCR plates or larger arrays of vessels). However other vessels may also be used. When distributing single cells into a large number of single vessels (e.g. 384 well plates), a population of single cells is obtained. Such a distribution may be performed, for example, by dispensing a volume into a single vessel that on average encompasses a cell concentration of one, 0.5 or 0.3 cell, thereby obtaining vessels that predominantly contain a single cell or less. Since distribution of cells by limiting dilution is a statistical event, a fraction of the vessels will be empty, a major fraction will contain a single cell, and a minor fraction will contain two or more cells. Where two or more cells are present in a vessel some scrambling of the variable region encoding sequences may occur among the cells present in the vessel. However, since it is a minor event it will not affect the overall utility of the present invention. Additionally, combinations of variable region encoding sequences which do not posses the desired binding affinity and specificity will most likely not be selected and hence these will likely be eliminated during a screening process. Therefore, minor events of scrambling will not significantly affect the final library of the present invention.

There are alternatives to cell distribution by limiting dilution using, for example, cell sorters such as FACS machines or robots that can be programmed to accurately dispense single cells into single vessels. These alternatives are preferable, since they are less laborious and are more efficient at uniformly obtaining a distribution of single cells into single vessels.

The enrichment, sorting and isolation procedures described above are performed such that the majority of the cells are kept intact. Rupture of cells during enrichment and sorting might result in scrambling of the variable region encoding sequences. However, this is not expected to be a problem since the frequency of rupture is expected to be low. Washing and possible RNAse treatment of the cells prior to distribution into single vessels will remove any RNA that has leaked during the process.

Further, when considering the above descriptions of how to distribute cells in order to obtain a population of single cells in a population of single vessels, it is as described above not essential that every vessel must contain a single cell. Rather, it will be clear to persons skilled in the art that the invention relies on a majority of the vessels containing single cells, and only a relatively small proportion of the vessels containing more than one cell, e.g. the number of vessels with two or more cells is preferably below 25% of the total amount of cells distributed, and more preferably below 10%, such as below 5%.

In a preferred embodiment, reverse transcription (RT) is performed using template derived from cells distributed individually among a plurality of vessels.

When the final distribution of the single cells to their single vessels has been performed, the single cells may be expanded in order to obtain populations of isogenic cells prior to reverse transcription. This process yields more mRNA to be used as template, which might be important if a rare target is to be amplified and linked. However, the cells should remain genetically identical with respect to the target gene during the expansion. The isolated cells or the population of isogenic cells can either be kept intact or lysed, as long as the template for the reverse transcription is not degraded. Preferentially, the cells are lysed in order to ease the following reverse transcription and PCR amplification.

In a different embodiment, multiplex overlap-extension RT-PCR or multiplex RT-PCR followed by linkage by ligation or recombination may also be utilized on template derived from a genetically diverse population of cells which have not been separated into single vessels, but which remain together as a pool of cells. This method may be used for the generation of combinatorial libraries. Such an approach will not require the distribution of single cells. However, the cells which may be used in this approach are the same as those described for the single cell approach, for example a population (pool) of sorted B lymphocytes. When performing the single-step multiplex overlap-extension RT-PCR or single-step multiplex RT-PCR followed by linkage by ligation or recombination on such a population of cells, it is preferable to lyse the cells prior to the reaction, and if desired total RNA or mRNA may be isolated from the lysate.

The sensitivity of the single-step multiplex overlap-extension RT-PCR of the present invention enables the use of a very low amount of template, e.g. an amount of template corresponding to the lysate of a single cell.

### Amplification and Linkage

The invention utilizes a variant of PCR in which two or more target sequences are amplified simultaneously in the same vessel, by including more than one set of primers, for example all the primers necessary to amplify variable region encoding sequences, in the same reaction. Generally this approach is known as multiplex polymerase chain reaction (multiplex PCR). The target sequences that are amplified by multiplex PCR according to the invention are linked, e.g. by overlap extension PCR, in close proximity to the amplification process. In particular, cognate pairs of antibody variable region encoding sequences are linked by this process.

One embodiment of the present invention exploits the fact that a multiplex primer mix can be designed to work in an overlap-extension PCR procedure, resulting in a simultaneous amplification and linkage of nucleotide sequences of interest. This multiplex overlap-extension PCR technique serves to reduce the number of reactions necessary to isolate and link nucleotide sequences of interest, in particular cognate pairs of linked variable region encoding sequences.

Other embodiments of the present invention apply linkage by ligation or by recombination as an alternative to linkage by multiplex overlap-extension PCR. In these procedures, the linkage is not performed simultaneously with the multiplex PCR amplification, but as a separate step following the amplification. However, linkage can still be performed in the same vessel as the multiplex PCR was performed in.

A multiplex overlap-extension PCR requires the presence of two or more primer sets (a multiplex primer mix), where at least one primer of each set is equipped with an overlap-extension tail. The overlap-extension tails enable the linkage of the products generated by each of the primer sets during amplification. Multiplex overlap-extension PCR differs from conventional overlap-extension PCR in that the sequences to be linked are generated simultaneously in the same vessel, thereby providing immediate linkage of the target sequences during amplification, without any intermediate purification.

In a preferred embodiment, a reverse transcription (RT) step precedes the multiplex PCR or multiplex overlap-extension PCR amplification, utilizing a template derived from an isolated single cell or a population of isogenic cells.

In a preferred embodiment, the invention uses nucleotide sequences derived from an isolated single cell or a population of isogenic cells as a template for the multiplex PCR amplification. Preferably, RNA from a single cell is reverse transcribed into cDNA prior to the multiplex PCR. For the amplification of some nucleic acid sequences of interest genomic DNA may be used as an alternative to mRNA. By using isolated single cells, or a population of isogenic cells derived by clonal expansion of an isolated single cell, as a template source, it is possible to avoid scrambling of nucleotide sequences derived from different cells within a population of cells. This is of importance when the aim is to maintain the original composition of the sequences of interest. Especially for the generation of a cognate pair of antibody variable region encoding sequences, the use of an isolated single cell or a population of isogenic cells as the template source is an important feature of the invention.

Additionally, the present invention facilitates the generation of libraries of linked nucleic acid sequences of interest, in particular combinatorial libraries and libraries of cognate pairs of variable regions.

As described elsewhere herein, one embodiment of the present invention encompasses a method of producing a library of cognate pairs comprising linked variable region encoding sequences, by providing a lymphocyte-containing cell fraction from an avian donor, optionally enriched for a particular lymphocyte population from said cell fraction, or wherein a particular lymphocyte population has been isolated from said cell fraction, and obtaining a population of isolated single cells by distributing cells from the lymphocyte-containing cell fraction or the enriched cell fraction individually among a plurality of vessels. Multiplex molecular amplification (e.g. multiplex RT-PCR amplification) of the variable region encoding sequences contained in the population of isolated single cells is performed and linkage of pairs of variable region encoding sequences is effected, wherein an individual pair of variable region sequences is derived from a single cell from the population. This technique may comprise two additional optional steps: in the first step the individual isolated single cell is expanded to a population of isogenic cells prior to performing multiplex RT-PCR amplification, thereby obtaining a plurality of vessels containing a diverse population of isogenic cells (one population of isogenic cells per vessel). The second optional step encompasses performing an additional amplification of the linked variable region encoding sequences.

As also described elsewhere herein, another embodiment of the invention encompasses the linkage of a plurality of non-contiguous nucleotide sequences of interest, by amplifying, in a multiplex PCR or multiplex RT-PCR amplification procedure, nucleotide sequences of interest using a template derived from an isolated single cell or a population of isogenic cells, and effecting linkage of the amplified nucleotide sequences of interest. This method may comprise an optional step of performing an additional amplification of the linked products.

In a preferred embodiment, an individual member of said library of cognate pairs comprising an immunoglobulin light chain variable region encoding sequence is associated with an immunoglobulin heavy chain variable region encoding sequence originating from the same cell.

The multiplex RT-PCR amplification of the invention can be performed either as a two-step process, where reverse transcription (RT) is performed separate from the multiplex PCR amplification (or alternatively multiplex molecular amplification), or as a single-step process, where the RT and multiplex PCR amplification steps are performed with the same primers in a single vessel.

The reverse transcription (RT) is performed with an enzyme having reverse transcriptase activity, resulting in the generation of cDNA from total RNA, mRNA or target specific RNA from an isolated single cell. Primers that can be utilized for the reverse transcription are for example oligo-dT primers, random hexamers, random decamers, other random primers, or primers that are specific for the nucleotide sequences of interest.

The two-step multiplex RT-PCR amplification procedure allows for the cDNA generated in the RT step to be distributed to more than one vessel, permitting storage of a template fraction before proceeding with the amplification. Additionally, the distribution of cDNA to more than one vessel allows for the performance of more than one multiplex PCR amplification of nucleic acid derived from the same template. Although this results in an increased number of separate reactions, it makes it possible to decrease the complexity of the multiplex primer mix if so desired.

In the single-step multiplex RT-PCR procedure, reverse transcription and multiplex PCR amplification are carried out in the same vessel. All the components necessary to perform both the reverse transcription and the multiplex PCR are initially added to the vessels and the reaction is performed. Generally, there is no need to add additional components once the reaction has been started. The advantage of single-step multiplex RT-PCR amplification is that it reduces the number of steps necessary to generate the linked nucleotide sequences of the invention. This is particularly useful when performing multiplex RT-PCR on an array of single cells, where the same reaction needs to be carried out in a plurality of vessels. Single-step multiplex RT-PCR is performed by utilizing the reverse primers present in the multiplex primer mix needed for the multiplex PCR amplification as primers for the reverse transcription as well. Generally, the composition needed for the single-step multiplex RT-PCR comprises a nucleic acid template, an enzyme with reverse transcriptase activity, an enzyme with DNA polymerase activity, a deoxynucleoside triphosphate mix (dNTP mix comprising dATP, dCTP, dGTP and dTTP) and a multiplex primer mix. The nucleic acid template is preferably total RNA or mRNA derived from an isolated single cell either in a purified form, as a lysate of the cell or still within the intact cell. Generally, the exact composition of the reaction mixture requires some optimization for each multiplex primer mixture to be used with the present invention. This applies to both the two-step and the single-step multiplex RT-PCR procedures.

For some single-step multiplex RT-PCR reactions it may be an advantage to add additional components during the reaction, for example addition of the polymerase following the RT step. Other components could for example be a dNTP mixture or a multiplex primer mix, possibly with a different primer composition. This can then be considered as a one-tube multiplex RT-PCR, which generally has the same advantages as the single-step multiplex RT-PCR, since it also limits the number of tubes necessary to obtain the desired linked products.

The nucleotide sequences of interest to be amplified by the multiplex RT-PCR procedure can be linked to one another by several methods, such as multiplex overlap-extension RT-PCR, ligation or recombination, using different multiplex primer mixes. Preferably, the multiplex RT-PCR amplification and linkage process is a single step or a two step process. However, the linkage process may also be performed as a multi step process, using for example a stuffer fragment to link the nucleic acid sequences of interest, either with PCR, ligation or recombination. Such a stuffer fragment may contain cis-elements, promoter elements or a relevant coding sequence or recognition sequence. In a preferred embodiment the linkage process is performed in the same vessel as the multiplex RT-PCR amplification.

In one embodiment, the linkage of a plurality of non-contiguous nucleotide sequences of interest is performed in association with the multiplex PCR amplification, utilizing a multiplex overlap-extension primer mix. This results in the combined amplification and linkage of the target sequences. Generally, the composition needed for the multiplex overlap-extension PCR comprises a nucleic acid template, an enzyme with DNA polymerase activity, deoxynucleoside triphosphate mix (dNTP mix comprising dATP, dCTP, dGTP and dTTP) and a multiplex overlap-extension primer mix.

In a particular embodiment of the present invention, the linkage of a plurality of non-contiguous nucleotide sequences of interest is performed by multiplex overlap-extension RT-PCR using a template derived from an isolated single cell or a population of isogenic cells, optionally with a step of performing an additional molecular amplification of linked products. Preferably, the multiplex overlap-extension RT-PCR is performed as a single-step/one-tube reaction.

A multiplex overlap-extension primer mix of the present invention comprises at least two primer sets capable of priming the amplification and linkage of at least two variable region encoding sequences, for example, amplification and linkage of sequences from immunoglobulin heavy chain variable region families with kappa or lambda light chain variable region families.

In another embodiment, the plurality of nucleotide sequences of interest, amplified by multiplex RT-PCR, are linked by ligation. To achieve this, the multiplex primer mix used for the multiplex RT-PCR is designed such that the amplified target sequences can be cleaved with appropriate restriction enzymes and covalent linkage by DNA ligation can be performed (the primer design is described in the section “Primer Mixtures and Design”). Following multiplex RT-PCR amplification with such a multiplex primer mix, the restriction enzymes needed to form compatible ends of the target sequences are added to the mixture together with the ligase. No purification of the PCR products is needed prior to this step, although purification may be performed. The reaction temperature for the combined restriction cleavage and ligation is approximately between 0 and 40° C. However, if the polymerase from the multiplex PCR reaction is still present in the mixture, an incubation temperature below room temperature is preferred, most preferred being temperatures between 4 and 16° C.

In yet another embodiment, the plurality of nucleotide sequences of interest, amplified by multiplex RT-PCR, are linked by recombination. In this approach, the amplified target sequences can be joined using identical recombination sites. Linkage is then performed by adding the recombinases that facilitate recombination. Suitable recombinase systems are, for example, Flp recombinase together with a variety of FRT sites, Cre recombinase together with a variety of lox sites, integrase φC31, which carries out recombination between the attP site and the attB site, the β-recombinase-six system and the Gln-gix system. Linkage by recombination has been exemplified for two antibody-encoding nucleotide sequences (V_{H} linked with V_{L}) (Chapal, N. et al. 1997 BioTechniques 23, 518-524).

In a preferred embodiment, the nucleotide sequences of interest comprise variable region encoding sequences and the linkage generates a cognate pair of variable region encoding sequences. Such a cognate pair may comprise one or more constant region encoding sequences in addition to the variable regions. In the latter case, the constant regions may be of human origin and the variable region cognate pair of avian origin, or the variable regions may be human sequences derived from a transgenic chicken or other transgenic birds. In the context of the present invention, such human sequences derived from a transgenic chicken are considered to be “avian-derived”.

More preferably, the nucleotide sequences of interest comprise immunoglobulin variable region encoding sequences and the linkage generates a cognate pair of light chain variable region and heavy chain variable region encoding sequences. Such a cognate pair may comprise one or more constant region encoding sequences in addition to the variable regions, and may e.g. be isolated from cells of the B-lymphocyte lineage enriched from a lymphocyte-containing cell fraction, such as whole blood, mononuclear cells or white blood cells as described above.

In another embodiment, the invention utilizes multiplex RT-PCR with a population of genetically diverse cells as a template source. The majority of heteromeric protein encoding sequences do not vary from cell to cell as is the case with variable region encoding sequences from binding proteins such as antibodies. Thus, when utilizing the present invention for the cloning of such non-variable heteromeric protein encoding sequences there is no need to perform an initial isolation of single cells.

In this embodiment, a plurality of non-contiguous nucleotide sequences of interest are linked randomly by a method comprising performing multiplex RT-PCR amplification of nucleotide sequences of interest using a template derived from a population of genetically diverse cells and effecting linkage of the amplified nucleotide sequences of interest. Further, the method may comprise an optional step of performing an additional amplification of the linked products. As with the single cell approach, the linkage can either be performed utilizing a multiplex overlap-extension primer mix for the amplification or alternatively by ligation or recombination. Preferably, the template derived from the population of cells is not strictly contained within the cells. The population of cells may for example be lysed.

Application of the process of random linkage on a population of cells expressing variant binding proteins allows for a simplified generation of combinatorial libraries of variable region encoding sequences. Preferably, the population of cells constitutes cells that express variable region binding proteins, such as B lymphocytes, splenocytes, cells isolated from bursa fabricii, hybridoma cells, plasma cells, plasmablasts, or a mixture of these cells.

The population of cells in the above-mentioned embodiment can for example be permeabilized or lysed, without additional purification, or the template nucleic acids can be isolated from the cells by standard procedures. The single-step multiplex RT-PCR procedure is preferred. However, the two-step procedure may also be used in this embodiment.

An efficient way to increase the specificity, sensitivity, and yield of the multiplex RT-PCR-linkage process is by performing an additional molecular amplification of the linked nucleotide sequences obtained from the multiplex RT-PCR followed by linkage by ligation or recombination or by multiplex overlap-extension RT-PCR. This additional amplification is preferably performed with PCR amplification, utilizing a primer mix adapted for amplifying the linked nucleic acid sequences of interest. The primer mix utilized may be the outer primers of the multiplex primer mix or multiplex overlap-extension primer mix, meaning the primers which anneal to the outermost 5′ end and 3′ end of the sense strand of the linked variable region encoding sequences, thereby enabling the amplification of the entire linked product. The outer primers can also be described as the primers of the multiplex overlap-extension primer mixture that do not contain overlap extension tails. Alternatively, a nested or semi-nested primer set can be used for the additional amplification of the linked nucleotide sequences. Such a nested PCR especially serves to increase the specificity of the method as well as to increase the amount of linked product. For the present invention, semi-nested PCR (as described in the section entitled Primer Mixtures and Design) is considered to function as well as nested PCR. Thus, it is desired although not necessary for the present invention to perform an additional PCR amplification of the linked products from the multiplex overlap-extension RT-PCR or of the products linked by ligation or recombination, preferably using nested PCR or semi-nested PCR.

The additional amplification can either be performed directly using a fraction of or the entire reaction product of multiplex overlap-extension RT-PCR, ligation or recombination, or using partially purified linked products from any one of these reactions, e.g. by performing an agarose gel electrophoresis of the linked products, and excising the fragment corresponding to the expected size of the linked variable region encoding sequences. For products linked by multiplex overlap-extension RT-PCR, the additional amplification is preferably performed directly on a fraction from the multiplex overlap-extension RT-PCR reaction, since this will assist linkage of the individual target sequences that may not have been linked in the first reaction.

### Primer Mixtures and Design

The primer mixtures of the present invention comprise at least four primers that form primer sets two by two, and which are capable of amplifying at least two different target sequences of interest. Primer sets include one or more primer pairs designed to amplify gene family variants. Mixtures of two or more of such primer pairs or primer sets constitute a multiplex primer mix. Antibody diversity in the chicken is achieved through gene conversion, a process whereby upstream pseudogenes for the heavy chain (HC) and light chain (LC) variable regions function as donors of sequences that are inserted into the single VH and VL gene by homologous recombination. This means that all variable regions can in principle be amplified to by a single primer pair for VH and a single primer pair for VL. In a preferred embodiment, a single VH and VL 5′ primer is used together with one or more 3′ constant region primers in the multiplex reaction, while a nested PCR reaction is performed with a single JH primer and a single JL primer. Preferably, a multiplex primer mix comprises at least 2, 3, 4, 5, 6, 7, 8, 9, 10, 11, 12, 13, 14, 15, 16, 17, 18, 19 or 20 primer pairs, for example at least 30, 40, 50, 60, 70, 80, 90 100, 110, 120, 130, 140 or 150 primer pairs. In particular for the amplification of variable region encoding sequences, an individual primer set within the multiplex primer mix may comprise more than two primer pairs. Preferably, an individual primer set comprises at least 3, 4, 5, 6, 7, 8, 9, 10, 11, 12, 13, 14, 15, 20, 25, 30, 35, 45, 50, 60, 70, 80, 90, 100, 110, 120, 130, 140, 150, 160, 170, 180, 190, 200, 220, 240, 260, 280 or 300 primers. Preferably, the total number of primers in a multiplex primer mix is at least 4, 5, 6, 7, 8, 9, 10, 11, 12, 13, 14, 15, 20, 25, 30, 35, 45, 50, 60, 70, 80, 90, 100, 125, 150 or 200 and at the most 225, 250, 275, 300, 325, 350, 375 or 400 primers.

All the primers of the present invention comprise a gene-specific region, and some primers are additionally equipped with a primer tail at the 5′ end of the primer, i.e. 5′ non-coding sequences which are fused to the 3′ end of the gene-specific primer part. Such a primer tail is approximately from 6 to 50 nucleotides long, but it may also be longer if desired. Upon amplification the primer tails are added to the target sequences.

Primer tails of the present invention are for example cloning tails and linkage tails, such as tails adapted for linkage by ligation, tails adapted for linkage by recombination, or overlap-extension tails.

Cloning tails may be from 6 to 20 nucleotides long or longer and comprise restriction sites and/or recombination sites which are useful for the insertion of the linked product into an appropriate vector.

To enable linkage by ligation, the primer sets of the multiplex primer mix are designed such that one part (forward or reverse primer(s)) of the first primer set is equipped with a linkage-tail containing a restriction site that upon cleavage will be compatible with a restriction site located in the linkage tail of one part of the second primer set. For linkage of more than two target sequences, the second part of the second primer set is equipped with a restriction site that upon cleavage will be compatible with a restriction site located in one part of the third primer set. This second restriction site located in the second primer set should be non-compatible with that of the first primer set. A considerable number of target sequences can be linked by designing primer sets this way. Restriction sites with a low frequency or which do not occur in the target sequences should be chosen. Further, it is preferable that compatible restriction sites are not identical, such that the site of ligation becomes cleavage-resistant for the particular restriction enzymes used. This will drive the reaction towards linkage of a first target sequence and a second target sequence, since linkage between identical target sequences will be cleavable by the restriction enzymes. Suitable pairs of restriction sites are, for example, SpeI with XbaI (alternatively NheI or AvrII can substitute for one or both of these), NcoI with BspHI, EcoRI with MfeI, or PstI with NsiI. For linkage, SpeI can for example be located in a first target sequence, XbaI can be located in a second target sequence, NcoI can be located at the other end of the second target sequence and BspHI in a third target sequence and so forth. To simplify the process further, it is an advantage if the restriction enzymes function in the same buffer.

To enable linkage by recombination, the primer sets of the multiplex primer mix can for example be designed as exemplified by Chapal et al. (1997 BioTechniques 23, 518-524), which is hereby incorporated by reference.

To enable the linkage of the nucleotide sequences of interest in the same step as the multiplex PCR amplification, tails adapted for overlap-extension PCR are added to at least one primer of each primer set of the multiplex primer mix, thereby generating a multiplex overlap-extension primer mixture.

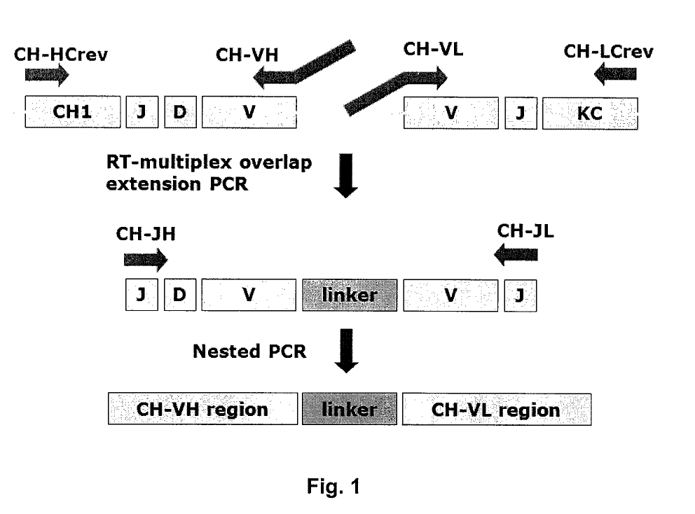

The overlap-extension tails are typically longer, ranging from 8 to 75 nucleotides in length, and may contain restriction sites or recombination sites which allow for subsequent insertion of regulatory elements such as promoters, ribosomal binding sites, termination sequences, or linker sequences such as in a scFv. The overlap-extension tail may also contain a stop codon if desired. Generally, there are three types of overlap-extension tails, as illustrated in FIG. 1 of WO 2005/042774. In type I the overlap-extension tails of two primer sets overlap solely with each other. The nucleotides of two overlap-extension tails need not all be complementary to each other. In one embodiment the complementary nucleotides represent between 60 to 85% of the overlap-extension tail. In type II overlap-extension tails, 4 to 6 of the 5′ nucleotides are complementary to the gene-specific region of the adjacent target sequence. In type III overlap-extension tails, the entire overlap is complementary to the adjacent target sequence. Type I and II overlap-extension tails are preferred when regulatory elements and the like are later to be inserted between the linked target sequences. Type II overlap-extension tails are preferred if the target sequences are to be linked by a defined linker as seen with scFv. Type III overlap-extension tails are preferred if the target sequences are to be linked in-frame to each other.

Design of overlap-extension tails is dependent on sequence features such as length, relative GC content (GC %), presence of restriction sites, palindromes, melting temperature, the gene-specific part to which they are coupled etc. The length of the overlap-extension tails should be between 8 and 75 nucleotides long, preferably they are from 15 to 40 nucleotides long. Even more preferred they are from 22 to 28 nucleotides long. The use of very long overlap-extension tails (50 to 75 nucleotides) could favor the linkage of the products produced by each primer set. However, the ratio between the length of the overlap-extension tail and the gene-specific region probably will need to be adjusted when using very long overlap-extension tails. The GC % preference is dependent on the length of the overlap-extension tail. Since shorter tails have a shorter stretch where they are complementary they need a higher GC % to strengthen the interaction than longer tails. Other principles of primer design should likewise be observed, e.g. primer dimerization and hairpin formation should be minimized, as should false priming. Further, it is known that Taq DNA polymerase often adds an adenosine (A) at the 3′ end of the newly synthesized DNA strand, and this can be accommodated for in overlap-extension tail design by enabling overlap-extension tails to accommodate 3′ non-template A addition.

The choice of primers that carry the linkage tail, e.g. the overlap-extension tail, or a tail adapted for linkage by ligation or by recombination, defines the order and direction of linkage of the target sequences. It is not critical to the invention whether it is the forward primer(s) or reverse primer(s) of a primer set or possibly both forward and reverse primers that are equipped with the linkage tail. However, this should still be given some consideration since the order and direction of the target sequences in the final product might be of relevance, e.g. for the insertion of regulatory elements such as promoters and termination sequences or for the in-frame linkage of the individual target sequences.

For the linkage of two nucleotide sequences of interest the linkage tail may be added either to the reverse primer(s) or forward primer(s) of each primer set used for the PCR amplification of each target sequence.

The present disclosure exemplifies addition of overlap-extension tails and tails adapted for linkage by ligation to the chicken VH and chicken VL forward primers of each set. This results in a linking direction of the products that is 5′ to 5′ (head-to-head and bi-directional). However, linkage tails can also be added to the reverse primer(s) of each set. This results in a linking direction of the product that is 3′ to 3′ (tail-to-tail and bi-directional). A third option is adding the linkage tails to the reverse primer(s) of the first primer set and the forward primer(s) of the second primer set or visa versa. This results in a 3′ to 5′ orientation (head-to-tail and uni-directional).

When linking more than two nucleotide sequences of interest some of the primer sets should have linkage tails on both the forward and reverse primers, such that one tail is complementary to a tail of the preceding primer set and the other tail is complementary to one of the primers of the subsequent primer set. This principle holds for all the primer sets that amplify target sequences that are to be linked between two other target sequences.

The design of the gene-specific primer part generally should observe known primer design rules such as minimizing primer dimerization, hairpin formation and non-specific annealing. Further, multiple G or C nucleotides as the 3′ bases are to be avoided when possible. The melting temperature (Tm) of the gene-specific regions in a primer set should preferably be equal to each other plus/minus 5° C. In the present invention Tm values between 45° C. and 75° C. are desirable and Tm values of about 60° C. are optimal for most applications. Advantageously, the initial primer design can be aided by computer programs developed for this task. However, primer designs generally need laboratory testing and routine optimization. This may be done, for example, by analyzing size, restriction fragment length polymorphism (RFLP) and sequencing of the amplification products obtained using the primer sets. The use of degenerate positions within primers is a useful approach when amplifying sequences with variable regions or when searching for new family members belonging to a specified class of proteins. The numbers of degenerate positions may also require optimization.

One feature of the present invention is primer mixes composed of at least two primer sets that are able to prime amplification and promote linkage of at least two nucleotide sequences of interest. The primer mixes of the present invention are capable of priming the amplification of at least two subunits or domains from heteromeric proteins, e.g. belonging to the class of enzymes, inhibitors, structural proteins, toxins, channel proteins, G-proteins, receptor proteins, immunoglobulin superfamily proteins, transportation proteins etc, preferably immunoglobulins.

A further feature of the present invention is use of a multiplex overlap-extension primer mix comprising primer sets wherein at least one primer set member of each primer set comprises an overlap-extension tail capable of hybridizing to the overlap-extension tail of a primer set member of a second primer set.

The overlap-extension tails enable the immediate linkage of the nucleotides of interest during the multiplex overlap-extension PCR amplification by equipping each individual product arising from the primer sets with a tail that is complementary to an adjoining product. This, however, does not mean that the linkage necessarily occurs during this first PCR amplification. Depending on the reaction setup, the majority of the actual linkages may be performed during an additional amplification with the outer primers of the first PCR amplification (multiplex PCR amplification).

Single primers can be used for the 5′ end of heavy and light chain variable region. Single primers complementary to heavy chain and light chain constant regions can be used as 3′ primers. Alternatively, light chain joining region primers may be used as reverse primers instead of the constant region primers. Alternatively, forward primers annealing in the UTR region preceding the leader sequence of the variable light and heavy chain may be used.

One embodiment of the present invention involves primers which anneal in the 3′ end of the leader encoding sequence preceding a variable region encoding sequence, and their use for amplification of variable region encoding sequences.

In one embodiment, the multiplex overlap-extension primer mix utilized for the multiplex overlap-extension PCR and possibly for the reverse transcription step as well comprises:
a) at least one chicken light chain constant region primer or one chicken light chain J-region primer complementary to the sense strand of an immunoglobulin light chain region encoding sequence;
b) one light chain V-region primer complementary to the antisense strand of an immunoglobulin light chain variable region encoding sequence or light chain variable region leader sequence, and capable of forming a primer set with the primer(s) in a);
c) at least one chicken heavy chain constant region primer, one chicken heavy chain primer complementary to the 3′ non-coding region of the mRNA, or one heavy chain J-region primer complementary to the sense strand of an immunoglobulin heavy chain domain encoding sequence; and
d) one chicken heavy chain V-region primer complementary to the antisense strand of an immunoglobulin heavy chain variable region encoding sequence or heavy chain variable region leader sequence, and capable of forming a primer set with the primer(s) in c).

In a further embodiment, the immunoglobulin light chain V-region and heavy chain V-region primers carry linkage tails, preferably in the form of complementary overlap-extension tails. This generates variable region encoding sequences that are linked in a head-to-head fashion. For the linkage of variable region encoding sequences in a head-to-tail fashion, either the chicken light chain constant region or chicken light chain J region and chicken heavy chain V region primers or both contain linkage tails or the chicken light chain V region primer and chicken heavy chain primer complementary to the 3′ non-coding region of the mRNA, the chicken heavy chain primer complementary to the heavy chain constant region or the primer complementary to the chicken heavy chain J region primers contain linkage tails or both, preferably in the form of complementary overlap-extension tails. For the linkage of variable region encoding sequences in a tail-to-tail fashion, the primer complementary to the chicken light chain constant or J region and the primer complementary to the chicken heavy chain constant or J region primers contain linkage tails, preferably in the form of complementary overlap-extension tails.

The present invention also encompasses primers for an additional PCR amplification of the linked products obtained by multiplex RT-PCR followed by linkage by ligation or recombination or by multiplex overlap-extension RT-PCR. This additional PCR amplification can be performed using a primer mix adapted for amplifying the linked target sequences. Such a primer mix may comprise the outer primers of the multiplex primer mix or multiplex overlap-extension primer mix, meaning the primers that anneal to the outermost 5′ end and 3′ end of the sense strand of the linked nucleotide sequences, thereby selectively enabling the amplification of the entire linked product. This process generally serves to increase the amount of linked product obtained from the multiplex RT-PCR followed by linkage by ligation or recombination or from the multiplex overlap-extension RT-PCR.

Alternatively, a primer set which is nested compared to the outer primers used in the primary multiplex RT-PCR or multiplex overlap-extension RT-PCR reaction can be used for the additional amplification of the linked nucleotide sequences. Such a primer set is termed a nested primer set. The design of nested primers generally observes the same design rules as for the gene-specific primers previously described, except that they prime partly or totally 3′ to the annealing position of the outer primers used in the multiplex RT-PCR or multiplex overlap-extension RT-PCR. The product resulting from a nested PCR may therefore be shorter than the linked product obtained by the multiplex RT-PCR followed by linkage by ligation or recombination or by multiplex overlap-extension RT-PCR. In addition to increasing the amount of linked product, the nested PCR further serves to increase the overall specificity, especially of the multiplex overlap-extension RT-PCR technology. However, it should be noted that not all multiplex primer mixes/multiplex overlap-extension primer mixes that have been described previously are suitable for combination with a nested primer set when performing the additional amplification. In such cases the outer primers of the multiplex primer mix/multiplex overlap-extension primer mix can be used for the additional amplification or a semi-nested PCR can be used.

In one embodiment, a mixture of J_{L} and J_{H} primers is used as nested primers for the additional amplification of the linked immunoglobulin variable region encoding sequences.

Nested primer sets of the present invention can also comprise one or more reverse (or forward) outer primer(s) from the first multiplex primer mix/multiplex overlap-extension primer mix and a second nested primer that primes 3′ to the annealing position of the forward (or reverse) outer primer(s) of the first multiplex primer mix/multiplex overlap-extension primer mix. The use of such a primer set for an additional PCR amplification is generally known as semi-nested PCR. Semi-nested PCR can for example be applied if it is difficult to design a nested primer in one specific region e.g. for the variable region sequences, because such a primer would have to anneal in the complementarity determining regions (CDRs). Further, semi-nested PCR can be used when it is desirable to keep one end of the linked sequences intact, e.g. for cloning purposes.

### Optimization of Multiplex Overlap-Extension PCR

The parameters of the multiplex overlap-extension PCR step of both the two-step and the single-step procedure can be optimized for several parameters (see, for example, Henegariu, O. et al. 1997. BioTechniques 23, 504-511; Markoulatos, P. et al. 2002. J. Clin. Lab. Anal. 16, 47-51). Generally the same optimization parameters apply for multiplex RT-PCR, although the ratio between outer and inner primers is less important for such a reaction.

a. Primer Concentration

The concentration of the primers carrying the overlap-extension tail (for example the V_{H} and V_{L} primers) is preferably lower than the concentration of the outer primers without the overlap-extension tail (for example J_{H} and light chain primers).

If one of the target sequences amplifies with a lower efficiency than the others, for example, as a result of a higher GC %, it may be possible to equalize the amplification efficacy. This may be done by using a higher concentration of the primer set which mediates amplification with low efficiency, or lowering the concentration of the other primers. For example, sequences encoding heavy chain variable regions tend to have a higher GC % and hence lower amplification efficiency than light chain variable regions. This points towards using V_{L} primers at a lower concentration than the V_{H} primers.

Further, when using a large number of primers the total primer concentration might be an issue. The upper limit may be determined experimentally by titration experiments. For the AmpliTaq Gold® PCR system from Applied Biosystems the upper limit was found to be 1.1 μM total oligonucleotide concentration, although for other systems it may however be as high as about 2.4 μM. Such an upper limit of total oligonucleotide concentration influences the maximal concentration of individual primers. If the individual primer concentration is too low it is likely to cause a poor PCR sensitivity.

The quality of the oligonucleotide primers has also been found to be important for the multiplex overlap-extension PCR. HPLC-purified oligonucleotides have produced the best results.

b. PCR Cycling Conditions:

The cycling conditions are preferably as follows, with 30-80 PCR cycles:

| | Time | Temperature | Note |
|---|---|---|---|
| Denaturation: | 10-30 s | 94° C. | |
| Annealing: | 30-60 s | 50-70° C. | (1) |
| Extension: | 1 min × EPL | 65-72° C. | (2) |
| Final extension: | 10 min | 65-72° C. | |

Notes:
(1) The annealing temperature is approximately 5° C. below the Tm of the primers.
(2) EPL is Expected Product Length in kB.

For the single-step multiplex overlap-extension RT-PCR the following steps were built into the cycling program prior to the amplification cycling outlined above:

| | Time | Temperature | Note |
|---|---|---|---|
| Reverse transcription: | 30 min | 42-60° C. | (1) |
| Polymerase activation: | 10-15 min | 95° C. | (2) |

Notes:
(1) These conditions are also used where separate reverse transcription is performed.
(2) Hot-start polymerases are favored in single-step RT-PCR. Activation according to manufacturer.

It is possible to optimize all these parameters. Especially the annealing temperature is important. Thus, initially all the individual primer sets that are to constitute the final primer mix should be tested separately in order to identify optimal annealing temperature and time, as well as elongation and denaturing times. This will give a good idea about the window within which these parameters can be optimized for the multiplex overlap-extension primer mix.

Problems with poor PCR sensitivity, for example due to low primer concentration or template concentration, can be overcome by using a high number of thermal cycles, meaning between about 35 and 80 cycles, preferably around 40 cycles. Further, longer extension times can improve the multiplex overlap-extension PCR process, i.e. extension times of about 1.5-5 min×EPL compared to the normal 1 min extension.

c. Use of Adjuvants

Multiplex PCR reactions can be significantly improved by using a PCR additive, such as DMSO, glycerol, formamide or betaine, which relaxes DNA, thus making template denaturation easier.

d. dNTP and MgCl₂

Deoxynucleoside triphosphate (dNTP) quality and concentration is important for the multiplex overlap-extension PCR. The best dNTP concentration is between 200 and 400 μM of each dNTP (dATP, dCTP, dGTP and dTTP), above which the amplification is rapidly inhibited. Lower dNTP concentrations (100 μM of each dNTP) suffice to achieve PCR amplification. dNTP stocks are sensitive to thawing/freezing cycles. After three to five such cycles, multiplex PCR often does not work well. To avoid such problems, small aliquots of dNTP can be made and kept frozen at −20° C.

Optimization of Mg²⁺ concentration is important, since most DNA polymerases are magnesium-dependent enzymes. In addition to the DNA polymerase, the template DNA primers and dNTPs bind Mg²⁺. Therefore, the optimal Mg²⁺ concentration will depend on the dNTP concentration, template DNA, and sample buffer composition. If primers and/or template DNA buffers contain chelators such as EDTA or EGTA, the apparent Mg²⁺ optimum may be altered. Excessive Mg²⁺ concentration stabilizes the DNA double strand and prevents complete denaturation of DNA, which reduces yield. Excessive Mg²⁺ can also stabilize spurious annealing of primer to incorrect template sites, thereby decreasing specificity. On the other hand, an inadequate Mg²⁺ concentration reduces the amount of product.

A good balance between dNTP and MgCl₂ is approximately 200 to 400 μM dNTP (of each) to 1.5 to 3 mM MgCl₂.

e. PCR Buffer Composition

Generally, KCl-based buffers suffice for multiplex overlap-extension PCR. However, buffers based on other components such as (NH₄)₂SO₄, MgSO₄, Tris-Cl, or combinations thereof may also be optimized to function with the multiplex overlap-extension PCR. Primer pairs involved in the amplification of longer products work better at lower salt concentrations (e.g. 20 to 50 mM KCl), whereas primer pairs involved in the amplification of short products work better at higher salt concentrations (e.g. 80 to 100 mM KCl). Raising the buffer concentration to 2× instead of 1× may improve the efficiency of the multiplex reaction.

f. DNA Polymerase

The present invention is exemplified with Taq polymerase. Alternatively, other types of heat-resistant DNA polymerases including, for example, Pfu, Phusion, Pwo, Tgo, Tth, Vent or Deep-vent, may be used. Polymerases without or with 3′ to 5′ exonuclease activity may either be used alone or in combination with each other.

### Vectors and Libraries

The linkage of nucleotide sequences of interest according to the present invention produces a nucleotide segment comprising the linked nucleotide sequences coding for variable regions of immunoglobulins. Further, libraries of such linked nucleic acid sequences are produced by the methods of the present invention, in particular libraries of non-human variable region encoding sequences linked or spliced to human constant region (heavy and light chain) sequences, or libraries of human variable region encoding sequences derived from a transgenic chicken or other transgenic bird linked to human constant region sequences.

In one embodiment, a segment containing linked nucleotide sequences of interest, or a library of such linked nucleotide sequences of interest, generated by a method of the present invention, is inserted into suitable vectors. The libraries may be combinatorial libraries or more preferably libraries of cognate pairs of variable region encoding sequences. The restriction sites generated by the outer primers, nested primers or semi-nested primers are preferably designed to match appropriate restriction sites of the vector of choice. The linked nucleic acid sequences of interest can also be inserted into vectors by recombination if one of the semi-nested, nested primers or outer primers is equipped with a suitable recombination site and the vector of choice contains one as well.

There are no limitations to the vectors that can be used as carriers of the products generated by one of the multiplex RT-PCR-linkage methods of the present invention. Vectors of choice may be those suitable for amplification and expression in cells including, for example, bacteria, yeast, other fungi, insect cells, plant cells, or mammalian cells. Such vectors may be used to facilitate further cloning steps, shuttling between vector systems, display of the product inserted into the vector, expression of the inserted product and/or integration into the genome of a host cell.

Cloning and shuttle vectors are preferably bacterial vectors. However, the other types of vectors may also be applied in cloning and shuttle procedures.

Display vectors can for example be phage vectors or phagemid vectors originating from the class of fd, M13, or f1 filamentous bacteriophages. Such vectors are capable of facilitating the display of a protein including, for example, a binding protein or a fragment thereof, on the surface of a filamentous bacteriophage. Display vectors suitable for display on ribosomes, DNA, yeast cells or mammalian cells are also known in the art. These comprise for example viral vectors or vectors encoding chimeric proteins.

Expression vectors exist for all the mentioned species and the suitable vector for any given situation depends on the protein to be expressed. Some expression vectors are additionally capable of integrating into the genome of a host cell either by random integration, or by site-specific integration, utilizing appropriate recombination sites. Expression vectors may be designed to provide additional encoding sequences that, when the linked product is inserted in-frame into these sequences, enable the expression of a larger protein, e.g. a full-length monoclonal antibody, in an appropriate host cell. This in-frame insertion may also facilitate the expression of chimeric proteins that are displayed on the surface of a filamentous bacteriophage or cell. In a bacteriophage display system, the linked nucleotide sequences of interest may be inserted in-frame to a sequence encoding a coat protein such as pIII or pVIII (Barbas, C. F. et al. 1991. Proc. Natl. Acad. Sci. USA 88, 7978-7982; Kang, A. S. et al. 1991. Proc. Natl. Acad. Sci. USA 88, 4363-4366).

In one embodiment, the individual segments of linked nucleotide sequences of interest comprise an immunoglobulin heavy chain variable region encoding sequence of avian origin associated with a light chain variable region encoding sequence of avian origin, inserted into a vector that contains sequence(s) encoding one or more human immunoglobulin constant domains, preferably both human light and heavy chain constant regions. The insertion is engineered such that the linked heavy chain variable region and/or light chain variable region encoding sequences are inserted in-frame with the constant region encoding sequences. Such an insertion can for example generate a Fab or F(ab′)₂ expression vector, a full-length antibody expression vector or an expression vector encoding a fragment of a full-length antibody. Preferentially such a vector is an expression vector suitable for expression (e.g. E. coli, phagemid, or mammalian vectors) and the constant region heavy chain encoding sequences are chosen from the human immunoglobulin classes IgGl, IgG2, IgG3, IgG4, IgM, IgA1, IgA2, IgD, or IgE, thereby enabling the expression of a Fab or full-length recombinant antibody. In addition to the constant heavy chain encoding sequences the vector may also contain a constant light chain encoding sequence chosen from human lambda or kappa chains. This is preferred in the generation of chimeric antibodies, as the linked nucleotide sequences in these cases only encode the immunoglobulin variable region encoding sequences (Fv′s) from the avian species.

In an alternative embodiment, the human constant region encoding sequence(s) is/are spliced or linked to the avian variable regions in a step of the molecular amplification procedure, by adding to the vessels a human constant region encoding sequence having an overlap with the avian sequence and appropriate primers ensuring the amplification of both variable and constant region(s) in frame. In this way the human constant kappa or lambda chain and/or a human constant heavy chain may be added. By using this procedure there is no need for providing a restriction site within the coding sequence, which is an advantage.

In one embodiment, a dual promoter cassette may be inserted into the expression construct, the dual promoter cassette being capable of directing the simultaneous expression of heavy and light chains, for example a bidirectional dual promoter cassette. The dual promoter cassette may further include a nucleic acid sequence coding for signal peptides for the heavy and light chains. The expression vector backbone may comprise a human constant light chain encoding sequence or a fragment thereof and/or a human constant heavy chain encoding sequence or a fragment thereof in order to produce chimeric avian/human antibodies.

Libraries of cognate pairs of the present invention may be introduced into vectors by two different approaches. In the first approach, the single cognate pairs are inserted individually into a suitable vector. This library of vectors may then either be kept separate or be pooled. In the second approach, all the cognate pairs are pooled prior to vector insertion, followed by mass-insertion into suitable vectors, generating a pooled library of vectors. Such a library of vectors comprises a large diversity of pairs of variable region encoding sequences.

In one embodiment the invention provides a library of antibodies with cognate pairs of linked variable region encoding sequences. Preferably, the individual antibodies of the library comprise an immunoglobulin light chain variable region encoding sequence associated with a heavy chain variable region encoding sequence from one avian species and human constant regions. A further embodiment is a sub-library selected from a parent library of cognate pairs of variable region encoding sequences as described throughout the application. A preferred embodiment of the present invention is a library or sub-library encoding cognate pairs of full-length chimeric immunoglobulins selected from human immunoglobulin classes IgA1, IgA2, IgD, IgE, IgG1, IgG2, IgG3, IgG4, or IgM. The libraries may comprise at least 5, 10, 20, 50, 100, 1000, 10⁴, 10⁵ or 10⁶ different cognate pair antibodies.

In a further embodiment of the present invention, said libraries of cognate pairs of linked variable region encoding sequences are obtainable by a method comprising the steps described herein. This library is also termed the parent library.

### Screening and Selection

The parent library of pairs of linked variable region encoding sequences isolated from a donor, utilizing one of the methods of the present invention, is expected to represent a diversity of binding proteins of which some will be irrelevant, i.e. not binding to a desired target, in particular for combinatorial libraries. Therefore, the present invention encompasses enrichment and screening for a sub-library encoding a subset of diversities of binding specificities directed against a particular target.

For libraries of cognate pairs the diversity of the library is expected to represent the diversity present in the donor material, with only a minor number of randomly linked variable regions. Thus, an enrichment step may not be necessary prior to the screening for target-specific binding affinities in a library composed of cognate pairs.

In a further embodiment, the method of generating a library of pairs of linked variable region encoding sequences further comprises creating a sub-library by selecting a subset of pairs of linked variable region sequences that encode binding proteins with a desired target specificity. Such a selection of linked variable region encoding sequences is also termed a library of target-specific cognate pairs.

In a preferred embodiment, the library of target-specific cognate pairs of variable region encoding sequences is transferred to an expression vector. The expression vector may be a mammalian expression vector, an insect cell expression vector, a yeast expression vector, a fungal expression vector, a plant expression vector or a bacterial expression vector, depending on the type of cell used for screening. Preferably the expression vector is mammalian.

Immunological assays are generally suitable for the selection of target-specific immunoglobulin variable region encoding sequences. Such assays are well know in the art and constitute for example FMAT, FLISA, ELISPOT, ELISA, membrane assays (e.g. Western blots), arrays on filters, or FACS. The assays can either be performed in a direct manner, utilizing the polypeptides produced from the immunoglobulin variable region encoding sequences, or alternatively, the immunoassays can be performed in combination with or following enrichment methods such as phage display, ribosome display, bacterial surface display, yeast display, eukaryotic virus display, RNA display or covalent display (reviewed in FitzGerald, K., 2000. Drug Discov. Today 5, 253-258). Both cognate Fab expression libraries and cognate full-length antibody expression libraries can be subjected to screening, thereby generating a sub-library of positive clones. Such screening assays and enrichment procedures are also suitable for Fv or scFv fragments or combinatorial libraries of linked variable regions.

In addition to immunological screening, a special feature of the invention is that it enables the use of various types of functional screening to select antibody secreting clones with desired properties. Such screening assays include but are not limited to proliferation assays, virus inactivation assays, cell killing assays, etc. Preferably the functional assays are carried out in high-throughput format using supernatants from cells transfected with expression vectors of the invention.

In a preferred embodiment, the selection of a sub-library of target-specific cognate pairs or combinatorial pairs of variable region encoding sequences is performed by using a high-throughput screening assay. High-throughput screening assays include, but are not restricted to, ELISA assays, functional assays performed with semi-automated or fully automated equipment.

When a sub-library of cognate pairs or combinatorial pairs of antigen-binding clones has been selected by an appropriate technology, it is possible to perform an additional analysis by DNA sequencing of the linked immunoglobulin light chain variable region and heavy chain variable region encoding sequences. Such DNA sequencing provides information about the library diversity and maturation within the CDR regions, and enables selection of a set of clones with a broad diversity, leaving out repeated clones. DNA sequencing will also reveal mutations introduced during the isolation process.

Mutations may be created by Taq DNA polymerase and they are most easily identified in the constant region encoding sequences and can easily be eliminated. However, Taq induced mutations will also be present in the variable region encoding sequences, where they are indistinguishable from the naturally occurring somatic mutations, which are also the result of random mutations in the variable region encoding sequences. Considering that the mutations are non-systematic and only affect particular pairs in distinct ways, it appears reasonable to disregard such changes.

In a further embodiment, the sub-library of target-specific and possibly sequence analysed pairs of linked immunoglobulin light chain variable region and heavy chain variable region encoding sequences are transferred to a mammalian expression vector. Such a transfer can be performed into any of the vectors described in the previous section, enabling the expression of a full-length recombinant antibody. If the screening is performed with a mammalian cognate full-length antibody expression library such a transfer may not be needed.

### Host Cells and Expression

The libraries of the present invention can be transferred to vectors suitable for expression and production of proteins encoded from the linked nucleic acid sequences of interest, in particular variable region containing binding proteins or fragments thereof. Such vectors are is described in the Vectors and Libraries section, and provide for the expression of for example full-length antibodies, Fab fragments, Fv fragments, scFv, membrane bound or soluble TcRs or TcR fragments of a species of choice.

One feature of the present invention is the introduction into a host cell of a library or a sub-library of vectors of cognate pairs of linked variable region encoding sequences or a single clone encoding a cognate pair of linked variable region encoding sequences, for amplification and/or expression. Host cells can be chosen from bacteria, yeast, other fungi, insect cells, plant cells, or mammalian cells. For expression purposes mammalian cells, such as Chinese hamster ovary (CHO) cells, COS cells, BHK cells, myeloma cells (e.g., Sp2/0 cells, NS0), NIH 3T3, fibroblast or immortalized human cells such as HeLa cells, HEK 293 cells, or PER.C6 cells are preferred.

The introduction of vectors into host cells may be accomplished by a number of transformation or transfection methods known to those skilled in the art, including calcium phosphate precipitation, electroporation, various chemical methods such as lipofection, microinjection, liposome fusion, RBC ghost fusion, protoplast fusion, viral infection and the like. The production of monoclonal full-length antibodies, Fab fragments, Fv fragments and scFv fragments is well known.

Manufacturing technologies for the production of recombinant polyclonal antibodies or other recombinant polyclonal proteins are described in WO 2004/061104 and WO 2008/145133. The technology described in WO 2004/061104 involves the generation of a collection of cells suitable as a manufacturing cell line by site-specific integration of nucleic acid sequences encoding e.g. cognate pairs of antibody heavy and light chains. WO 2008/145133 describes a different approach for manufacturing recombinant polyclonal antibodies or other polyclonal proteins based on random integration of the individual genes of interest into host cells, preferably followed by cloning of single cells with desired characteristics. The individual cell clones, which each produce an individual member of the polyclonal protein, are then mixed in order to generate a polyclonal manufacturing cell line for the production of a polyclonal protein. Compared to the site-specific integration approach of WO 2004/061104, the random integration approach of WO 2008/145133 provides greater flexibility and can result in higher protein expression levels. Both approaches are advantageous, however, in that they have been found to allow stable production of polyclonal antibodies in a single batch, with uniform growth rates and expression levels over time and between batches.

The generation of a polyclonal manufacturing cell line and the production of a recombinant polyclonal protein from such a cell line can more generally be obtained by several different transfection and manufacturing strategies, as described e.g. in WO 2004/061104.

One way is to use a library of vectors mixed together into a single composition for the transfection of a host cell line with a single integration site per cell. This method is termed bulk transfection or transfection in bulk. Generally, the vector and host cell design will ensure that a polyclonal cell line capable of unbiased growth will be obtained upon appropriate selection. A frozen stock of the polyclonal cell line will be generated before initiation of the recombinant polyclonal protein manufacturing.

Another way is to use a library of vectors split into fractions, containing approximately 5 to 50 individual vectors of the library in a composition, for transfection. Preferably, a fraction of the library constitutes 10 to 20 individual vectors. Each composition is then transfected into an aliquot of host cells. This method is termed semi-bulk transfection. The number of aliquots transfected will depend on the size of the library and the number of individual vectors in each fraction. If the library for example constitutes 100 distinct cognate pairs, which are split into fractions containing 20 distinct members in a composition, 5 aliquots of host cells would need to be transfected with a library composition constituting a distinct fraction of the original library. The aliquots of host cells are selected for site-specific integration. Preferably, the distinct aliquots are selected separately. However, they can also be pooled before selection. The aliquots can be analyzed for their clonal diversity and only those with sufficient diversity will be used to generate a polyclonal cognate pair library stock. To obtain the desired polyclonal cell line for manufacturing, the aliquots can be mixed before generating the freezing stock, immediately after they have been retrieved from the freezing stock or after a short proliferation and adaptation time. Optionally, the aliquots of cells are kept separate throughout production, and the polyclonal protein composition is assembled by combining the products of each aliquot rather than the aliquots of cells before production.

A third way is a high throughput method in which host cells are transfected separately using the individual vectors constituting the library of cognate pairs. This method is termed individual transfection. The individually transfected host cells are preferably selected for site specific integration separately. The individual cell clones generated upon selection may be analyzed with respect to proliferation time and preferably, and those with similar growth rates are used to generate a polyclonal cognate pair library stock. The individual cell clones can be mixed to obtain the desired polyclonal cell line before generating the freezing stock, immediately after they have been retrieved from the freezing stock, or after a short proliferation and adaptation time. This approach may eliminate any possible residual sequence bias during transfection, integration and selection. Alternatively, the individually transfected host cells are mixed before selection is performed; this will enable control of sequence bias due to transfection.

A shared feature in the manufacturing strategies outlined above is that all the individual cognate pairs constituting the recombinant polyclonal protein can be produced in one bioreactor or a limited number of bioreactors. The only difference is the stage at which one chooses to generate the collection of cells that constitutes the polyclonal manufacturing cell line.

In one embodiment, the invention provides a population of host cells comprising a cognate library or sub-library of linked pairs of variable region encoding sequences. In a further embodiment, a population of host cells comprises a library obtained from a population of isolated single lymphocyte cells, utilizing the multiplex RT-PCR amplification followed by linkage by ligation or recombination or the multiplex overlap-extension RT-PCR technology of the present invention to link the cognate pairs.

In another embodiment, the invention provides a population of host cells comprising a combinatorial library or sub-library of linked pairs of variable region encoding sequences. A population of host cells according to the present invention will encompass a diverse population of cells corresponding to the diversity of the library the cells have been transformed/transfected with. Preferably, each cell of the population of cells only constitutes one cognate pair of the entire library of cognate pairs, and no individual member of the library of cognate pairs exceeds more than 50%, more preferred 25%, or most preferred 10%, of the total number of individual members expressed from the population of host cells.

The host cells are preferably mammalian cells.

A population of host cells as described above can be utilized for the expression of a recombinant polyclonal binding protein, since individual cells of the population comprise variable region encoding sequences of different diversity.

In one embodiment, the invention provides a recombinant polyclonal protein expressed from a population of host cells comprising a library of vectors encoding diverse cognate pairs of linked variable region encoding sequences, where such a library is obtainable by the method of the present invention. Typically, a recombinant polyclonal protein of the present invention comprises at least 2, 5, 10, 20 or 50 proteins composed of different cognate pairs.

The invention allows for expression of a recombinant polyclonal antibody from a population of host cells comprising a library of vectors encoding diverse cognate pairs of heavy chain variable region and light chain variable region encoding sequences.

A host cell obtained according to the method of the invention may also be used for to production of a monoclonal protein, in particular a monoclonal antibody comprising a cognate pair of a light chain variable region and a heavy chain variable region. Preferably, such a monoclonal production cell line is not a hybridoma cell line. Such a monoclonal antibody can be generated by adding the following steps to the method of linking a plurality of non-contiguous nucleotide sequences of interest a) inserting said linked nucleic acid sequences into a vector; b) introducing said vector into a host cell; c) cultivating said host cells under conditions suitable for expression; and d) obtaining the protein product expressed from the vector inserted into said host cell. Preferably, the vector introduced into the host cell encodes an individual cognate pair of variable region encoding sequences.

### Applications of the Invention

The use of recombinant monoclonal antibodies in diagnostics, treatment and prophylaxis is well known. Recombinant monoclonal and polyclonal antibodies generated by the present invention will be able to be used in the same manner as antibody products generated by existing technologies. In particular, a pharmaceutical composition comprising a polyclonal recombinant antibody as the active ingredient, in particular where the polyclonal recombinant antibody comprises cognate pairs of variable region encoding sequences, combined with at least one pharmaceutically acceptable excipient, can be produced by means of the present invention. The polyclonal recombinant antibody composition can be specific for or reactive against a predetermined disease target, and the composition can thus be used for the treatment, amelioration or prevention of diseases such as cancer, infections, inflammatory diseases, allergy, asthma and other respiratory diseases, autoimmune diseases, immunological malfunctions, cardiovascular diseases, diseases in the central nervous system, metabolic and endocrine diseases, transplant rejection, or undesired pregnancy, in a mammal such as a human, a domestic animal, or a pet.

All patent and non-patent references cited in the present application are hereby incorporated by reference in their entirety.

The invention will be further described in the following non-limiting examples.

### EXAMPLES

### Example 1

This example demonstrates different gating and sorting strategies for isolation of antibody-producing B cells from chickens or hens, hereafter referred to simply as chickens (Gallus gallus; Isa Warren strain) by fluorochrome conjugated antibody staining and sorting of cells using fluorescence activated cell sorting (FACS) using a combination of lymphocyte specific cell surface markers. Bu-1 is a well-known specific chicken B cell surface antigen that is present on B cells during the maturation to antibody producing cells, and is lost during differentiation to plasma cells (Rothwell et al. (1996) Vet. Immunology Immunopathology 55:225-34). In addition, antibody-secreting cells were detected based on the presence of IgY at the cell surface. Despite the fact that cell surface presentation of IgY is lost during differentiation to plasma cells, this direct marker for antibody expression was included in the sorting strategy based on an assumption that the membrane level of IgY allows single cell sorting as observed for mammalian IgG expression systems (Wiberg et al. (2006) Biotechnol. Bioeng. 94(2):396-405). T cell cells were detected and eliminated from the sorted populations by the presence of the CD3 antigen. The chickens used in this study were immunized with the tetanus toxoid (TT) antigen. Consequently, biotinylated tetanus toxoid was also used in combination with fluorochrome labelled streptavidin to stain and select the raised population of TT-specific cells. The selected B cell population was assayed for both antibody-producing B cells and specific anti-TT antibody producing cells by ELISpot assays. The spleen used as the source of the cell population comprised the most differentiated B-cells and thereby a high content of antibody secreting cells (Mansikka et al., 1989, Scand. J. Immunol. 29(3):325-331)).

### Immunizations:

Six 23 week old female chickens, Lohmann Brown Lite strain, were immunized by subcutaneous injection of 0.5 mg tetanus toxoid (TT) in complete Freund's adjuvant (CFA) and repeatedly boosted 14, 21 and 28 days after the primary immunization with 0.5 mg TT in incomplete Freund's adjuvant. Spleens from the immunized chickens were taken at 2, 7 and 10 days after the final boost immunization, and splenocytes were recovered immediately.

### Purification of Chicken Splenocytes:

A chicken was euthanized and the spleen was removed immediately. The spleen was briefly stored in 10 ml of 4° C. RPMI 1640 (Invitrogen, CA, US) with 1% Pencillin/streptomycin (P/S) (Invitrogen, CA, US) and kept on ice. The spleen tissue was transferred to a 70 μm cell strainer (BD Falcon™ 352350) in a 50 ml tube. The back of a 10 ml syringe plunger was used to macerate the cells through the filter, which during the procedure was rinsed at regular intervals with 4° C. complete medium (RPMI 1640 with 10% fetal calf serum (FCS) and 1% P/S). The cells in the suspension were harvested by centrifuged at 300×g at 4° C. for 5 minutes and subsequently washed by suspension in 50 ml 4° C. FACS buffer (2% FCS in Phosphate Buffered Saline (PBS)) and centrifuged as described previously. Finally, the cells were diluted in 4° C. FACS buffer and run through a 50 μm FACS filter (BD 340603) before being used for FACS or being stored at −140° C. in freezing medium (10% DMSO, 90% fetal calf serum).

Staining of splenocytes with the relevant markers to identity antibody-producing B cells: 50 μl of each of the murine anti-chicken antibodies as indicated in the list below were added to 1×10⁸ cells in 1 ml 4° C. FACS buffer and incubated for 20 minutes at 4° C. in the dark and washed twice after the primary staining and three times after the secondary staining.
Primary staining:
   Bu-1-FITC (Southern Biotech 8395-02)
   CD3-PECy5 (Abcam ab25537)
   IgY-PE (Southern Biotech 8320-09)
   Biotinylated tetanus toxoid
Secondary staining:
   Streptavidin-APC-CY7

The samples were analyzed on a FACSAria™ cell sorter using compensation on anti-mouse Igκ CompBeads (BD 51-90-9001229) with the above mentioned antibodies. The antibody-producing B cell population was identified by setting up various sorting gates, after which the sorted populations were tested in ELIspot assays (Example 2) and as templates for the Symplex™ PCR reactions (Example 3).
The applied sorting gates were as follows:
   1. Bu-1⁺CD3⁻
   2. Bu-1⁺CD3⁻IgY⁺
   3. Bu-1⁺CD3⁻IgY⁺TT⁺
   4. Intermediary population, P2
   5. P2 IgY⁺ (P3)
   6. P2 IgY⁺TT⁺ (P4)
   7. Bu-1⁻CD3⁻
   8. Bu-1⁻CD3⁻IgY⁺
   9. Bu-1⁻CD3⁻IgY⁺TT⁺

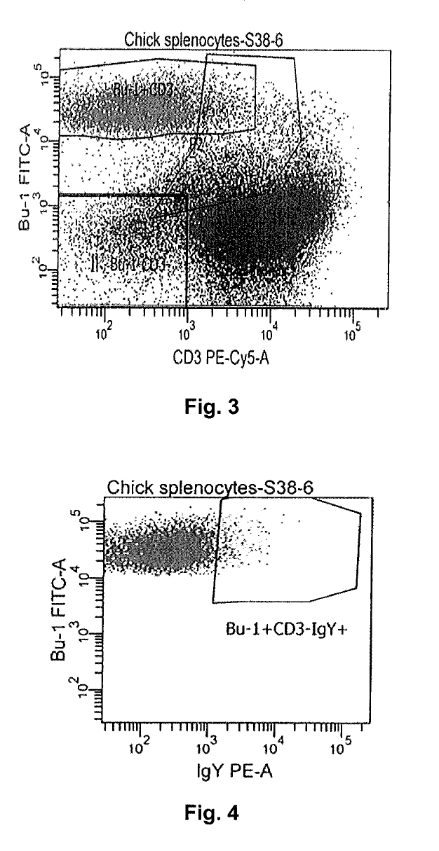

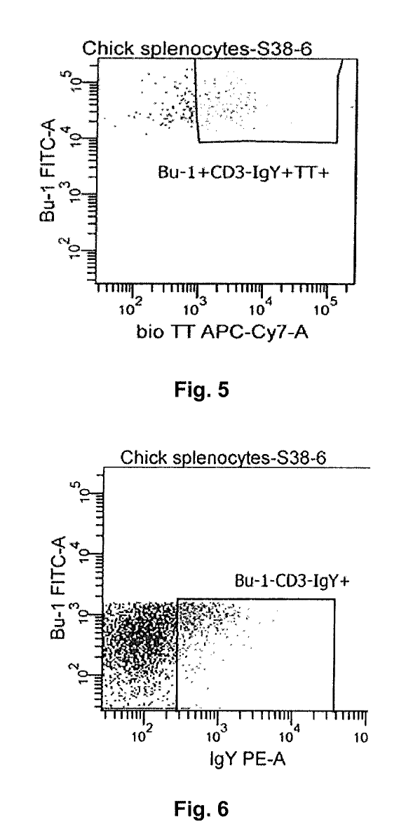

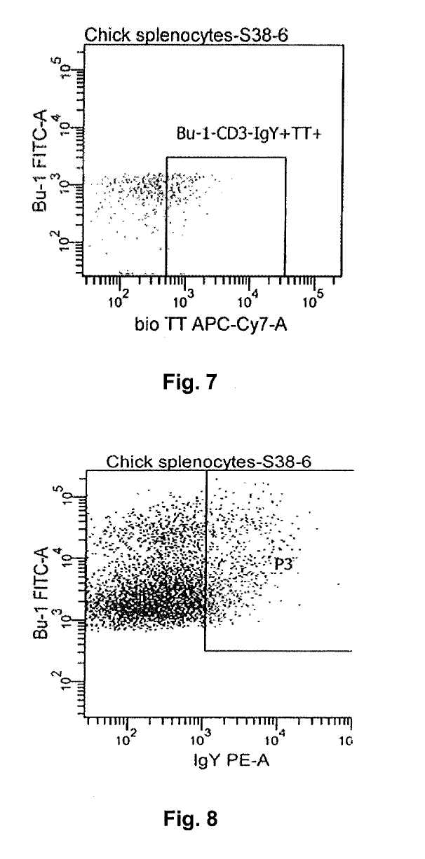

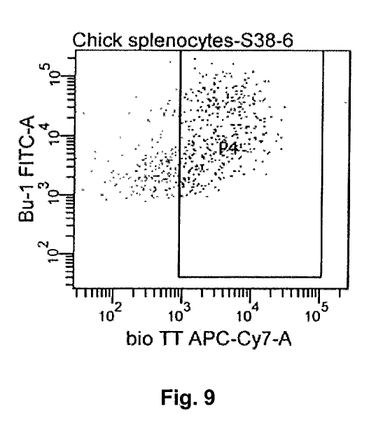

Sorting gates 1-3 are shown in FIG. 3, while sorting gates 4-9 are shown in FIGS. 4-9, respectively.

### Example 2

This example demonstrates that by the use of IgY-specific and TT-specific ELISpot assays the antibody-producing B cell populations can be identified among the sorted B cell populations in Example 1.

### Solutions:

Washing buffer (1×PBS, 0.05% Tween):
Blocking buffer: (RPMI, 2% skim milk)
Complete RPMI: (RPMI, 10% Inactivated FCS, 1% P/S)

### ELISpot Assay:

A PVDF-bottomed plate (Multiscreen-HTS, Millipore, MSIP S45 10) was coated with 100 μl anti-IgY antibody (Abcam ab 6872) or tetanus toxoid, both 10 μg/ml, diluted in PBS and incubated overnight at 4° C. Wells coated with PBS only were used as a negative control. The plates were washed 3 times in PBS and subsequently blocked with 200 μl blocking buffer at 4° C. for at least 2 hours. The buffer was then removed and replaced with 50 μl complete RPMI.

Ten thousand cells from populations 1, 4 and 7 (Example 1) were sorted in duplicate into the IgY, TT or PBS coated wells of the ELISpot plates. Two thousand IgY-positive cells from populations 2, 5 and 8 and five hundred TT-positive cells from populations 3, 6 and 9 were likewise sorted into ELISpot wells. The ELISpot plates were left overnight under standard cell incubation conditions to allow for antibody secretion.

After overnight incubation the plates were washed 6 times; 3 times in washing buffer and 3 times in PBS for removal of cells and unbound antibody. To detect the secreted and captured IgY or TT-specific IgY, 100 μl/well horse radish peroxidase (HRP) conjugated anti-IgY antibody (Abcam ab6877) diluted 10,000 times in blocking buffer was added, followed by incubation for 1 hour at 37° C. The washing procedure was repeated before 100 μl freshly made chromogenic substrate consisting of 0.015% H₂O₂ and 0.3 mg/ml of 3-amino-9-ethylcarbazole in 0.1 M sodium acetate 0.1 M acetic acid pH 5.1 was added. The reaction was stopped by washing with H₂O after 4 minutes of development. The number of spots was determined using a stereo microscope and the results are shown in Table 1.

TABLE 1
Characterization of the differently sorted populations by ELIspot and Symplex ™ PCR

| | | Bu-1⁺ | Bu-1⁺ | | | | | Bu-1⁻ | Bu-1⁻ |
|---|---|---|---|---|---|---|---|---|---|
| | Bu-1⁺ | CD3⁻ | CD3⁻ | | | | Bu-1⁻ | CD3⁻ | CD3⁻ |
| | CD3⁻ | IgY⁺ | IgY⁺TT⁺ | P2 | P3 | P4 | CD3⁻ | IgY⁺ | IgY⁺TT⁺ |
| % anti-TT- | 0.02 | 0 | 0 | 0.6 | 0.4 | 0.1 | 0 | 0.3 | 1.8 |
| ASCs | | | | | | | | | |
| % IgY-ASCs | 0.8 | 3.0 | 2.8 | 4.8 | 9.6 | 6.2 | 1.6 | 15.9 | 33.2 |
| % anti-TT- | 2 | 0 | 0 | 13 | 4 | 2 | 0 | 2 | 5 |
| ASCs of IgY- | | | | | | | | | |
| ASCs | | | | | | | | | |
| Symplex ™(1) | 2 | 1 | ND | 18 | 17 | ND | 2 | 22 | ND |

ELIspot data is the mean from two independent wells
ND: Not determined
(1)Number of positive Symplex ™ PCR reactions out of a total of 96 reactions (see Example 3 for details).

From the ELISpot assay data it can be concluded that the frequency of antibody-producing cells is optimal among Bu-1⁻CD3⁻IgY⁺ cells. The results from single-cell Symplex PCR reactions confirmed this finding.

### Example 3

### Cloning of a Chimeric Chicken-Human Anti-Tetanus Toxoid Antibody Repertoire

Chicken spleen B cell populations P2 and P3 as described in Example 1 were single cell sorted as described above. For the use in Symplex™ PCR the cells were sorted directly into four 96-well PCR plates containing 10 μl buffer (Qiagen OneStep RT-PCR kit) and stored at −80° C. until the RT-PCR reactions were performed.

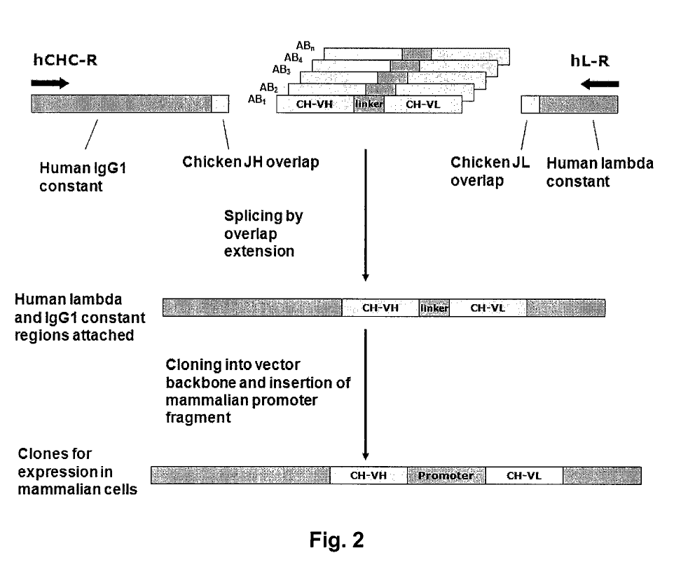

The chicken Symplex PCR amplification was performed on the four 96-well plates. The basic principles of the reactions are (FIGS. 1 and 2):
First an RT reaction is performed in which heavy and light chain cDNA synthesis is primed by specific constant region primers.
Secondly, a multiplex PCR reaction is performed using VH and VL 5′ region primers equipped with complementary overhangs facilitating the formation of connected VH and VL by overlap extension. 3′ primers are located in the constant region of the heavy and light chain sequences.
A nested PCR reaction is performed amplifying only conjugated VH and VK using JH and JL primers equipped with overhangs for the later addition by overlap extension of human lambda light chain and IgG1 heavy chain constant regions. The Symplex™ PCR product consists of approximately 700 nucleotides depending on the size of the CDRs.
Human IgG1 and lambda constant regions are appended by overlap extension.
The final reaction product consists of chicken VH coupled to human IgG1 constant region and chicken VL coupled to human lambda constant region conjugated 5′ end to 5′ end and connected with a linker. The linker region contains RE sites for insertion of a mammalian cell promoter-leader fragment, while flanking sites facilitate cloning.
The linked chimeric LC-HC fragments are cloned into a vector backbone and a mammalian cell promoter-leader fragment is inserted.

For the combined multiplex RT-PCR reaction the set of primers shown in Table 2 was used, employing the Qiagen OneStep RT-PCR kit essentially according to the manufacturer's instructions. PCR plates with sorted cells were thawed on ice. Enzymes, reaction buffer, dNTPs and primers were added to obtain total reaction volumes of 20 μl. Cycling conditions for the multiplex PCR reaction were:

$$55 ° C . , 30 min . \\ 95 ° C . , 10 min . \\ \begin{matrix}94 ° C . , 40 sec . \\ 60 ° C . , 40 sec . \\ 72 ° C . , 5 min .\end{matrix} \} 35 cycles$$

$$72 ° C . , 10 min .$$

TABLE 2
Primer set used for the combined RT and
multiplex reaction

| | Conc. | | Seq. |
|---|---|---|---|
| Primer | (nM) | Sequence | no. |
| CH-VH | 200 | TATTCCCATGGCGCGCCGCCGTGACGTTG | 1 |
| | | GACGAGTC | |
| CH-HCrev1 | 100 | AACAGGCGGATAGAGGGTAC | 2 |
| CH-HCrev2 | 100 | GAAGCTTTTCCTCTTCTCGC | 3 |
| CH-VL | 200 | GGCGCGCCATGGGAATAGCTAGCCGCGCT | 4 |
| | | GACTCAGCC | |
| CH-LCrev1 | 100 | TTGGTGGCTTCGTTCAGCTC | 5 |
| CH-LCrev2 | 100 | AAGTCGTTTATCAGGCACAC | 6 |

Conc. indicates the final concentration in the reactions

The nested PCR was performed with the set of primers shown in Table 3 using FastStart polymerase (Roche) and reagents essentially according to the manufacturer's instructions. 1 μl of the multiplex Symplex PCR product was used as template per nested reaction in a total volume of 20 μl. Reaction conditions were:

$$\begin{matrix}95 ° C . , 30 sec . \\ 60 ° C . , 30 sec . \\ 72 ° C . , 90 sec .\end{matrix} \} 35 cylces$$

$$72 ° C . , 10 min .$$

TABLE 3
Primer set used for the nested reaction

| | Conc. | | SEQ ID |
|---|---|---|---|
| Primer | (nM) | Sequence | NO: |
| CH-JH | 200 | GGAGGCGCTCGAGACGATGACTTCGGTCC | 7 |
| CH-JL | 200 | CTAGGACGGTCAGGGTTGTCC | 8 |

Conc. indicates the final concentration in the reactions

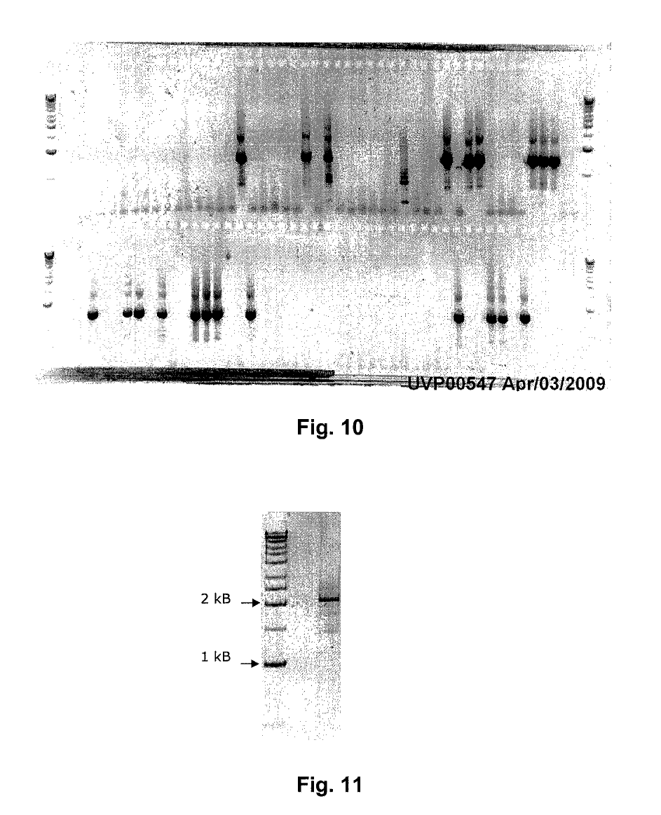

Finally, 10 μl of each final reaction product was analyzed on a 1% agarose gel. FIG. 10 shows an example from a 96-well plate with 21 reaction products with the expected electrophoretic mobility. A total of 90 bands resulted on the 4 plates. Similar analyses were performed on 96 single cells from all of the populations of B cells described in Example 1 defined by the various gates. The number of Symplex™ PCR reactions yielding the VH and VL overlap product from each subpopulation is given in Table 1 above.

Aliquots from all wells in the four 96-well PCR plates were pooled and the ˜700 by VH-VL band was purified on a 1% agarose gel. Human lambda light chain and IgG1 heavy chain constant regions were added by overlap extension PCR:
A human IgG1 constant heavy chain region cDNA fragment (codon optimized to improve expression) was amplified from an antibody expression plasmid with primers hCHC-F and hCHC-R (Table 4) using Phusion® polymerase (Finnzymes). The hCHC-F primer contains a 5′ part complementary to primer CH-JH used in the nested reaction. Primer hCHC-R introduces a flanking PacI site used for cloning of the overlap band. The ˜1000 kb constant region fragment was purified on a 1% agarose gel.
A human lambda light chain constant region fragment was amplified using primers hL-F and hL-R (Table 4) and an antibody expression plasmid as template. The hL-F primer contains a 5′ part complementary to primer CH-JL used in the nested reaction. The hL-R primer introduces a flanking NotI site used for cloning of the overlap band. The ˜350 kb constant region fragment was purified on a 1% agarose gel.
The purified VH-VL, the human lambda and the human IgG1 constant region bands were mixed (25:12.5:25 ng, respectively) and an overlap extension PCR was performed with Phusion® polymerase using primers hCHC-R and hL-R (Table 4). The reaction products (with the approximately 2 kb overlap band) are shown in FIG. 11.

TABLE 4
Primer set used for the addition of human
lambda light chain and IgG1 heavy chain
constant regions

| | Conc. | | SEQ ID |
|---|---|---|---|
| Primer | (nM) | Sequence | NO: |
| hCHC-F | 200 | GGACCGAAGTCATCGTCTCGAGTGCCAGCAC | 9 |
| | | CAAGGGCCCCTC | |
| hCHC-R | 200 | GGTCTAGAGTTAATTAATCACTTGCC | 10 |
| hL-F | 200 | AACCCTGACCGTCCTAGGTCAGCCCAAGGCC | 11 |
| | | AACCC | |
| HL-R | 200 | GGTTTAAACGCGGCCGCTTATTATGAACATT | 12 |
| | | CTGTAGGG | |

Conc. indicates the final concentration in the reactions

The fragment was digested with NotI and Pad and inserted into a plasmid with IRES-DHFR (internal ribosome entry site-dihydrofolate reductase) coupled 3′ to the heavy chain, and appropriate polyadenylation signals for LC and HC-IRES-DHFR, by ligation. E. coli TOP10 (Invitrogen) competent cells were electroporated and transformants were seeded on large (35×35 cm) LB-agar plates with 100 μg/ml carbenicillin. The resulting colonies were scraped off the plates and the bacterial pellet was used for the purification of DNA using a Maxi-Prep kit (Compact Prep, Qiagen). The purified DNA representing the antibody repertoire from the sorted cells was digested with AscI and NheI and a bidirectional promoter fragment with signal sequence-encoding regions for expression in mammalian cells was inserted by ligation. E. coli TOP10 cells were transformed by electroporation with the ligation mix and seeded on LB-agar plates as described above.

### Example 5

### Expression and Screening for Specific Anti-Tetanus Toxoid Antibodies in the Cloned Repertoire

Single E. coli colonies from Example 4 were picked into single wells of five 96 deep-well plates containing LB broth with 100 μg/ml carbenicillin and grown overnight at 37° C. in a shaker incubator. DNA was prepared from the 5 plates using a 96 Turbo Miniprep Kit (Qiagen) and the presence of VH-LC inserts was verified by colony PCR. Transient transfections were performed using the FreeStyle™ 293 cell expression system (Invitrogen). 100 μl of cells (10⁶/ml) in FreeStyle™ medium were seeded in five 96-well plates. Cells were transfected with miniprep DNA from five 96-well plates: 1 μl 293Fectin™ (Invitrogen) was diluted in 52 μl OptiMEM® medium (Invitrogen). A mean of 0.75 μg miniprep purified plasmid DNA was added and the mixture was incubated for 20 min. 7 μl of the mix was added per well and the 96-well plates were incubated at 37° C. at 5% CO₂ with shaking (150 rpm) for 4 days.

Maxisorp™ (Nunc) plates were coated overnight with tetanus toxoid (State Serum Institute, Copenhagen) in a concentration of 5 μg/ml. The plates were blocked with skim milk and the antibody-containing supernatants from the transient transfections were diluted 1:5 in buffer (PBS with Tween-20 and skim milk) before addition to the wells. Binding of antibody was detected by incubation with peroxidase-conjugated goat anti-lambda light chain antibody (Serotec) and peroxidase reaction using TMB Plus (KemEnTec) and A₄₅₀ was measured.

Using an arbitrary cut-off value of 2 times background, 11 supernatants could be considered positive for anti-tetanus reactivity. To further substantiate this, 7 of the 11 supernatants were tested in a new ELISA essentially as described above using tetanus toxoid-coated plates. The same 7 supernatants were tested in parallel against non-coated plates that were only blocked with skim milk as a negative control. The results are shown in Table 5. There is a clear binding to tetanus toxoid-coated wells, while there is no binding to non-coated wells.

TABLE 5
ELISA test of tetanus toxoid reactivity of
7 supernatants from transfected 293 cells

| | | Skim milk |
|---|---|---|
| Supernatant | Tetanus toxoid | block |
| Plate 1 - G8 | 1.30 | 0.04 |
| Plate 3 - H12 | 2.65 | 0.05 |
| Plate 4 - E10 | 0.97 | 0.05 |
| Plate 5 - A9 | 0.12 | 0.04 |
| Plate 5 - C9 | 1.64 | 0.04 |
| Plate 5 - E8 | 0.60 | 0.04 |
| Plate 5 - G4 | 2.41 | 0.04 |
| Buffer | 0.04 | 0.04 |

Supernatants from all wells in a single 96-well plate were tested for the presence of IgG in ELISA using a catching antibody against human Fc and a peroxidase-conjugated detecting antibody against human lambda chain. Using a cutoff value of 10 times background, more than 80% of the wells contained IgG lambda reactivity, which confirms expression of the chicken-human chimeric antibody.

### Example 6

### Sequence Analysis

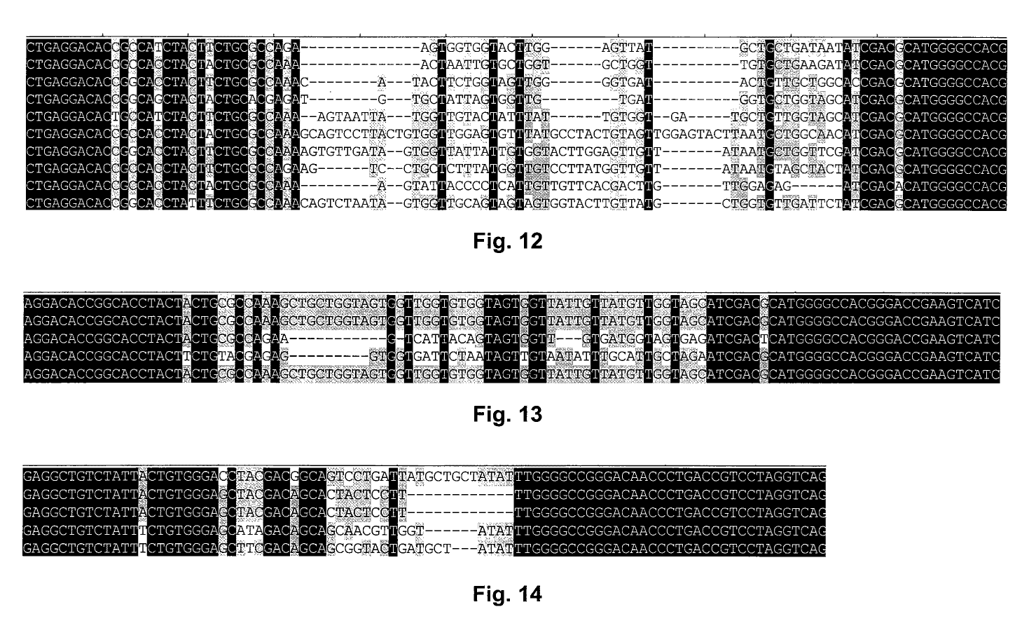

12 antibody clones from plate 1 in Example 5 were chosen randomly and VH and VL regions were sequenced. Overall, it appeared that the gene structure of the sequenced plasmids was as intended, with chicken-derived VH and VL situated head-to-head with the promoter fragment in between and with correctly appended human constant regions. FIG. 12 shows an alignment of the VH CDR3 regions with short stretches of flanking framework and human HC constant regions of 10 of these clones. The alignment illustrates that there is a high degree of diversity. Similar results were obtained for the chicken VL regions, which also show a high degree of diversity (data not shown).

SEQ ID NOs:13 to 22, respectively, in the appended sequence listing are the full VH sequences containing the 10 sequences shown from top to bottom in FIG. 12. SEQ ID Nos: 13 to 22 include the respective sequences between the AscI site (last part of the signal peptide-encoding sequence) and the XhoI site (linking chicken VH and human IgG1 constant region cDNA).

The 7 tetanus toxoid-positive clones from Example 5 were sequenced, and 5 of them produced reliable sequence data. The DNA sequences of VH and VL were aligned with the following results:
VH regions of three antibodies (number 1, 2 and 5, from the top in FIG. 13) were identical except for a single nucleotide difference, while the two others were very different.
VL regions of three antibodies (number 1, 2 and 3 from the top in FIG. 14) were identical over most of the sequence (frameworks, CDR1 and CDR2), except for 2 nucleotide bases, which may be due to PCR introduced mutations, while a single clone (number 1 from the top in FIG. 14) differed from the two others in the CDR3 sequence, indicating further differentiation in this region by gene conversion. The VL regions of the remaining clones (number 4 and 5 from the top in FIG. 14) were, as was the case for the VH regions, very different.

ELISA and sequence analysis combined showed that the 90 Symplex PCR bands after cloning gave at least 3 totally different tetanus-specific antibodies, thereby confirming that the method of the invention is suitable for identification of antigen-specific chicken-derived antibodies.

### Example 7

### Conclusions

Together, Examples 1 to 6 demonstrate that we have established a FACS-based method for production of a single cell sorted B cell population that is enriched for antibody secreting B cells, and that expressed antibody genes can be recovered from the single cells by the chSymplex™ PCR technology described herein. The population of chicken B lymphocytes (CD3⁻) can be divided into subpopulations based on the amount of Bu-1 and IgY at the cell surface, which allows for a significant enrichment of antibody secreting cells as demonstrated by results obtained by both IgY and TT specific ELIspot assays. The ELIspot results were further supported by a correlation with the increased frequency of positive Symplex™ PCR reactions, as shown in Table 1. Furthermore, the fact that TT-specific antibodies were identified from the pilot scale antibody repertoire that consisted of 90 Symplex PCR products demonstrates that isolation of antigen-specific chicken antibodies is readily achievable by the method of the invention.

1. A method for producing a library of cognate pairs comprising linked variable region encoding sequences, said method comprising:
a) providing a lymphocyte-comprising cell fraction from a donor of avian origin;
b) obtaining a population of isolated single cells by distributing cells from said cell fraction individually into a plurality of vessels, wherein at least a subpopulation of the cells expresses immunoglobulin genes and optionally any avian B cell marker antigen; and
c) amplifying and effecting linkage of the variable region encoding sequences contained in said population of isolated single cells by amplifying, in a multiplex molecular amplification procedure, nucleotide sequences of interest using a template derived from an isolated single cell or a population of isogenic cells, and effecting linkage of the amplified nucleotide sequences of interest.

2. The method according to claim 1, wherein the subpopulation of cells is characterized by any of the following:
expression of IgY (IgY⁺),
expression of IgY and CD3 negative (IgY⁺CD3⁻),
expression of IgY, no or low expression of Bu-1, and CD3 negative (IgY⁺Bu-1⁻CD3⁻),
expression of Bu-1 and IgY (Bu-1⁺IgY⁺),
expression of Bu-1 and IgY, and CD3 negative (Bu-1⁺IgY⁺CD3⁻),
expression of Bu-1 but not any monocyte markers (Bu-1⁺, monocyte⁻),
expression of Bu-1 and no or low levels of IgM (Bu-1⁺IgM⁻), or
expression of Bu-1 and BAFF (Bu-1⁺BAFF⁺).

3. The method according to claim 2, wherein the subpopulation of cells is IgY⁺.

4. The method according to claim 3, wherein the subpopulation of cells is IgY⁺CD3⁻.

5. The method according to claim 1, wherein individual isolated single cells in the population of single cells are expanded to populations of isogenic cells prior to performing amplification and linkage.

6. The method according to claim 1, wherein the lymphocyte-comprising cell fraction comprises splenocytes, whole blood, bone marrow, mononuclear cells, or white blood cells.

7. The method according to claim 1, wherein the lymphocyte-comprising cell fraction is enriched for plasma cells, plasmablasts or memory B cells.

8. The method according to claim 1, wherein the nucleotide sequences of interest comprise immunoglobulin variable region encoding sequences and the linkage generates a cognate pair of a light chain variable region encoding sequence associated with a heavy chain variable region encoding sequence.

9. A method of randomly linking a plurality of non-contiguous nucleotide sequences of interest, said method comprising:
a) amplifying, in a multiplex molecular amplification procedure, nucleotide sequences of interest using a template derived from a population of genetically diverse cells, wherein the genetically diverse cells are derived from a lymphocyte-comprising cell fraction from a donor of avian origin, and wherein at least a subpopulation of the cells expresses immunoglobulin genes and optionally any avian B cell marker antigen; and
b) effecting linkage of the nucleotide sequences of interest amplified in step a).

10-14. (canceled)

15. The method of claim 1, further comprising assessing prior to multiplex molecular amplification that the lymphocyte-comprising cell fraction comprises cells expressing detectable levels of IgY.

16. The method of claim 1, further comprising enriching said lymphocyte-comprising cell fraction for a lymphocyte population expressing IgY prior to multiplex molecular amplification.

17. The method of claim 1, further comprising isolating from said lymphocyte-comprising cell fraction cells expressing IgY, prior to multiplex molecular amplification.

18. The method of claim 1, comprising using an automated sorting procedure to enrich the lymphocyte-comprising cell fraction for a lymphocyte population expressing IgY, to isolate from the lymphocyte-comprising cell fraction cells expressing IgY, or both.

19. (canceled)

20. The method of claim 1, wherein the avian is a chicken.

21. (canceled)

22. The method of claim 20, wherein the chicken is transgenic and expresses human immunoglobulin sequences.

23. The method according to claim 1, wherein said multiplex molecular amplification procedure is a multiplex RT-PCR amplification performed as a two step process comprising a separate reverse transcription step prior to the multiplex PCR amplification, or in a single step comprising initially adding all the components necessary to perform both reverse transcription and multiplex PCR amplification into a single vessel.

24-26. (canceled)

27. The method according to claim 23, wherein said linkage of the nucleotide sequences of interest is effected in association with the multiplex RT-PCR amplification, utilizing a multiplex overlap-extension primer mix.

28. (canceled)

29. The method according to claim 1, wherein an additional molecular amplification, utilizing a primer mix adapted for amplifying the linked nucleic acid sequences of interest, is performed.

30. The method according to claim 1, further comprising inserting the linked nucleotide sequences or the library of cognate pairs into a vector.

31. (canceled)

32. The method according to claim 30, wherein the linked nucleotide sequences or the individual members of the library of cognate pairs comprise an immunoglobulin heavy chain variable region encoding sequence associated with light chain variable region encoding sequence and said sequences are inserted in-frame into a vector containing sequences encoding one or more immunoglobulin constant domains or fragments thereof.

33. The method according to claim 30, further comprising creating a sub-library by selecting a subset of cognate pairs of linked variable region sequences that encode binding proteins with a desired target specificity, thereby generating a library of target-specific cognate pairs of variable region encoding sequences.

34. The method according to claim 32, further comprising transferring said cognate pair of variable region encoding sequences to a mammalian expression vector.

35. The method according to claim 34, wherein the mammalian expression vector encodes one or more constant region domains selected from human immunoglobulin classes IgA1, IgA2, IgD, IgE, IgG1, IgG2, IgG3, IgG4, IgM, kappa light chain and lambda light chain.

36. The method according to claim 30, further comprising the steps:
a) introducing a vector encoding a segment of linked nucleotide sequences into a host cell;
b) cultivating said host cells under conditions adapted for expression; and
c) obtaining the protein product expressed from the vector introduced into said host cell.

37. The method according to claim 36 wherein said protein product is an antibody comprising a cognate pair of a light chain variable region associated with a heavy chain variable region.

38. A multiwell plate comprising in the majority of wells,
one cell derived from a lymphocyte-comprising cell fraction from an avian donor, said cell expressing immunoglobulin genes including IgY and/or Bu-1 antigen, and
buffers and reagents required for carrying out reverse transcription of mRNA and for amplifying heavy and light chain variable encoding regions.

39. A method for generating a vector encoding a chimeric antibody with human constant regions and non-human variable regions, said method comprising:
a) providing a lymphocyte-comprising cell fraction from a donor of avian origin;
b) obtaining a population of isolated single cells by distributing cells from said cell fraction individually into a plurality of vessels;
c) amplifying and effecting linkage of the variable region encoding nucleic acids contained in said population of isolated single cells by amplifying, in a multiplex molecular amplification procedure, said nucleic acids using a template derived from an isolated single cell or a population of isogenic cells; and effecting linkage of the amplified nucleic acids encoding variable regions of heavy and light chains;
d) effecting linkage of the amplified variable regions to human constant regions; and
e) inserting the obtained nucleic acid into a vector.

40-53. (canceled)

54. A library of vectors encoding chimeric antibodies, each chimeric antibody consisting of chicken immunoglobulin variable region encoding sequences and human immunoglobulin heavy and light chain constant regions.

55-62. (canceled)

63. A method for producing a library of avian-derived immunoglobulin variable region encoding sequences, said method comprising:
a) providing a lymphocyte-comprising cell fraction from a donor of avian origin;
b) obtaining a population of isolated single cells by distributing cells from said cell fraction individually into a plurality of vessels, wherein at least a subpopulation of the cells express immunoglobulin genes, and optionally at least one avian B cell marker antigen; and
c) amplifying the variable region encoding sequences contained in said population of isolated single cells by amplifying, in a multiplex molecular amplification procedure, nucleotide sequences of interest using a template derived from an isolated single cell or a population of isogenic cells.

64. (canceled)


SEQUENCE LISTING (32 sequences)

SEQ ID NO: 1 - DNA, 37 bp, Artificial Sequence
  Primer
tattcccatggcgcgccgccgtgacgttggacgagtc

SEQ ID NO: 2 - DNA, 20 bp, Artificial Sequence
  Primer
aacaggcggatagagggtac

SEQ ID NO: 3 - DNA, 20 bp, Artificial Sequence
  Primer
gaagcttttcctcttctcgc

SEQ ID NO: 4 - DNA, 44 bp, Artificial Sequence
  Primer
ggcgcgccatgggaatagctagccgcgctgactcagccgtcctc

SEQ ID NO: 5 - DNA, 20 bp, Artificial Sequence
  Primer
ttggtggcttcgttcagctc

SEQ ID NO: 6 - DNA, 20 bp, Artificial Sequence
  Primer
aagtcgtttatcaggcacac

SEQ ID NO: 7 - DNA, 21 bp, Artificial Sequence
  Primer
ctaggacggtcagggttgtcc

SEQ ID NO: 8 - DNA, 29 bp, Artificial Sequence
  Primer
ggaggcgctcgagacgatgacttcggtcc

SEQ ID NO: 9 - DNA, 43 bp, Artificial Sequence
  Primer
ggaccgaagtcatcgtctcgagtgccagcaccaagggcccctc

SEQ ID NO: 10 - DNA, 26 bp, Artificial Sequence
  Primer
ggtctagagttaattaatcacttgcc

SEQ ID NO: 11 - DNA, 36 bp, Artificial Sequence
  Primer
aaccctgaccgtcctaggtcagcccaaggccaaccc

SEQ ID NO: 12 - DNA, 39 bp, Artificial Sequence
  Primer
ggtttaaacgcggccgcttattatgaacattctgtaggg

SEQ ID NO: 13 - DNA, 362 bp, Gallus gallus
gccgtgacgttggacgagtccgggggcggcctccagacgcccggaggagcgctcagcctcgtctgcaagggctccgggttcaccttcagtgattacagcgtgcagtgggtgcgccaggtgcccggcaaggggctagagtgggtcgctggtattagcaacactgctattgagacaacatacggggcggcggtgcagggccgtgccaccatctcgagggacaacgggcagagcacagtgaggctgcagctgaacaacctcagggctgaggacaccgccatctacttctgcgccagaagtggtggtacttggagttatgctgctgataatatcgacgcatggggccacgggaccgaagtcatcgt

SEQ ID NO: 14 - DNA, 365 bp, Gallus gallus
gccgtgacgttggacgagtccgggggcggcctccagacgcccggaggagggctcagcctcgtctgcaaggcctccgggttcaccttcagcagttacagcatgcagtgggtgcgccaggcgcccggcaagggactggaatgggtcgctggtattgatagtaatggtggtagtaacacatactacggggcggcggtgaagggccgtgccaccatctcgagggacgatgggcagagcacagtgaggctgcagctgaacaacctcagggctgaggacaccgccacctactactgcgccaaaactaattgtgctggtgctggttgtgctgaagatatcgacgcatggggccacgggaccgaagtcatcgt

SEQ ID NO: 15 - DNA, 365 bp, Gallus gallus
gccgtgacgttggacgagtccgggggcggcctccagacgcccggaggagcgctcagcctcgtctgcaagggctccgggttcaccttcagcagttatgccatgggctggatgcgacaggcgcccggcaaggggctggagtatgtcgcgggtattagaaacactggtagtaggacatggtacggggcggcggtgaagggccgtgccaccatctcgagggacgacgggcagagcacagtgaggctgcagctgaacaacctcagggctgaggacaccggcacctacttctgcgccaaacatacttctggtagttggggtgatactgttgctggcaccgacgcatggggccacgggaccgaagtcatcgt

SEQ ID NO: 16 - DNA, 362 bp, Gallus gallus
gccgtgacgttggacgagtccgggggcggcctccagacgcccggaggagcgctcagcctcgtctgcaaggcctccgggttcaccttcagtgattatcagatgttctgggtgcgacaggctcccggcaaggggctggaatacgtcgctgctattgagaatgatggtagtgacacatggtacgggtcggcggtggatggccgtgccaccatctcgagggacaacgggcagagcacagtgaggctgcagctgaacaacctcagggctgaggacaccggcagctactactgcacgagatgtgctattagtggttgtgatggtgctggtagcatcgacgcatggggccacgggaccgaagtcatcgt

SEQ ID NO: 17 - DNA, 380 bp, Gallus gallus
gccgtgacgttggaccgaagtcatcgtctcgagcgcctccagacgcccgaaggagcgctcagccttgtctgcaaggcctccgggttcgacttcagcagttacgccatgaactgggtgtgacaggcgcccggcaaggggctggaatgggtcgctggtattgatgctgctggtaggtacacaaactacggggcagcggtgaagggccgtgccaccatctcgagggacaacgggcagagcacactgaggctgcagctgaacaacctcagggctgaggacactgccatctacttctgcgccaaaagtaattatggttgtactatttattgtggtgatgctgttggtagcatcgacgcatggggccacgggaccgaagtcatcgt

SEQ ID NO: 18 - DNA, 392 bp, Gallus gallus
gccgtgacgttggacgagtccgggggcggcctccagacgcccggaagagcgctcagcctcgtctgcaaggcctccgggttcaccttcagcacttacaacatattctgggtgcgacaggcgcccggcaaggggctggagttcgtcgcagctattgacaatactggtagctacacagcatacggggcagcggtgaagggccgtgccaccatctcgaaggacaacgggcagagcacagtgaggctgcagctgaggaacctcagggctgaggacaccgccacctactactgcgccaaagcagtccttactgtggttggagtgtttatgcctactgtagttggagtacttaatgctggcaacatcgacgcatggggccacgggaccgaagtcatcgt

SEQ ID NO: 19 - DNA, 383 bp, Gallus gallus
gccgtgacgttggacgagtccgggggcgacctccagacgcccggaggagcgctcagcctcgtctgcaaagcctccgggttcaccttcagcagttatgccatgggttggatgcgacaggcgcccggcaaggggctggagttcgtcgctggtatttatagcactggtagttacccaagctacgcgccggcagtgaagggccgtgccaccatctcgaaggacaacgggcagagcacagtgaggcttcagttgaacaacctcagggctgaggacaccggcacctacttctgcgccaaaagtgttgatagtggttattattgtggtacttggagttgttataatgctggttcgatcgacgcatggggccacgggaccgaagtcatcgt

SEQ ID NO: 20 - DNA, 377 bp, Gallus gallus
gccgtgacgttggacgagtccgggggcggcctccagacgcccggaggagggctcagcctcgtctgcaaggcctccggattctctattggcgcttacgacatgctctgggtgcgacagacgcccggcaaggggctggaatatgtcgcgggtattagcgacagtggtaaatacacatactacgcgccggcggtgcagggccgtgccaccatctcgagggacaacgggcagagcacagtgaggctgcagctgaacaacctcagggctgaggacaccgccacctacttctgcgccagaagtcctgctctttatggttgtccttatggttgttataatgtagctactatcgacgcatggggccacgggaccgaagtcatcgt

SEQ ID NO: 21 - DNA, 368 bp, Gallus gallus
gccgtgacgttggacgagtccgggggcggcctccagacgcccggaggagcgctcagcctcgtctgcaaggcctccgggttcgacttcagtggttacgccatgggttgggtgcgacaggcgcccggcaagggcctggaatacgtcggtgttattaacaatagtggtagtgtcatagactatgggtcggcggtgcagggccgtgccaccatctcgagggacaacgggcagagcacagtgaggctgcagctgaacaacctcagggctgaggacaccgccacctactactgcgccaaaagtattacccctcattgttgttcacgacttgttggagagatcgacacatggggccacgggaccgaagtcatcgt

SEQ ID NO: 22 - DNA, 383 bp, Gallus gallus
gccgtgacgttggacgagtccgggggcggcctccagacgcccggaggagcgctcagcctcgtctgcaaggcctccgggttcaccttcagcacttatggcatgatgtgggtgcgacaggcgcccggcaagggactggagtgggtcggaagtattatcaatgatggtagttggacaggctacgggtcggcggtgaagggccgtgccaccatctcgagggacaacgggcagagcacagtgaggctgcagctgaacaacctcagggctgaggacaccggcacctatttctgcgccaaacagtctaatagtggttgcagtagtagtggtacttgttatgctggtgttgattctatcgacgcatggggccacgggaccgaagtcatcgt

SEQ ID NO: 23 - DNA, 377 bp, Gallus gallus
gccgtgacgttggacgagtccgggggcggcctccagacgcccggaggagcgctcagcctcgtttgcaaggcctccgggctctctatcggcagatacgacatggtctgggtgcggcaggcgcccggcaaggggctggagtgggtcgctggtattagcaatagtggtagtagcacaggatatgggccggcggtgaagggccgtgccaccatctcgagggacaacgggcagagcacagtgaggctgcacctgaacaacctcagggctgaggacaccggcacctactactgcgccaaagctgctggtagtggttggtgtggtagtggttattgttatgttggtagcatcgacgcatggggccacgggaccgaagtcatcgt

SEQ ID NO: 24 - DNA, 377 bp, Gallus gallus
gccgtgacgttggacgagtccgggggcggcctccagacgcccggaggagcgctcagcctcgtttgcaaggcctccgggctctctatcggcagatacgacatggtctgggtgcgacaggcgcccggcaaggggctggagtgggtcgctggtattagcaatagtggtagtagcacaggatatgggccggcggtgaagggccgtgccaccatctcgagggacaacgggcagagcacagtgaggctgcacctgaacaacctcagggctgaggacaccggcacctactactgcgccaaagctgctggtagtggttggtgtggtagtggttattgttatgttggtagcatcgacgcatggggccacgggaccgaagtcatcgt

SEQ ID NO: 25 - DNA, 359 bp, Gallus gallus
gtgacgttggacgagtccgggggcggcctccagacgcccggaagagcgctcagcctcgtctgcaaggcctccgggttcaccttcagtgattatggcatgggctgggtgcgacaggcgcccggcaaagggctggagtgggtcgctggtattgatgatgatggtagttggacatcatatgggccggcggtgcagggccgtgccaccatctcgagggacgacgggcagagcacagtgaggctgcagctgaacaacctcagggctgaggacaccggcacctactactgcgccagaagtcattacagtagtggttgtgatggtagtgagatcgactcatggggccacgggaccgaagtcatcgt

SEQ ID NO: 26 - DNA, 368 bp, Gallus gallus
gccgtgacgttggacgagtccgggggcggcctccagacgcccggaggagcgctcagcctcgtctgcaagggctccgggttcaccttcagcagttacagcatgcagtgggtgcgacaggcgcccggcaacgggctggaatgggtcgcaactattagcagcagtggtagtagcacgtggtatgcggcggcggtgaagggccgtgccaccatctcgagggacaacgggcagagcacagtgaggctgcagctgaacaacctcagggctgaggacaccggcacctacttctgtacgagaggtggtgattctaatagttgtaatatttgcattgctagaatcgacgcatggggccacgggaccgaagtcatcgt

SEQ ID NO: 27 - DNA, 377 bp, Gallus gallus; identical to SEQ ID NO: 24

SEQ ID NO: 28 - DNA, 642 bp, Gallus gallus
gcgctgactcagccgtcctcggtgtcagcgaacccgggagaaaccgtcaagatcacctgctctggggatgacagctatggttatggctggttccagcagaagtctcctggcagtgcccctgtcactgtgatctatcacaacaacaacagaccctcggacatcccttcacgattctccggttccaaatccggctccacagccacattaaccatcactggggtccaagtcgaggacgaggctgtctattactgtgggacctacgacggcagtcctgattatgctgctatatttggggccgggacaaccctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 29 - DNA, 630 bp, Gallus gallus
gcgctgactcagccgtcctcggtgtcagcgaacccgggagaaaccgtcaagatcacctgctcttgggatgacagctatggttatggctggttccagcagaagtctcctggcagtgcccctgtcactgtgatctatcacaacaacaacagaccctcggacatcccttcacgattctccggttccaaatccggctccacagccacattaaccatcactggggtccaagtcgaggacgaggctgtctattactgtgggagctacgacagcactactccttttggggccgggacaaccctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 30 - DNA, 630 bp, Gallus gallus
gcgctgactcagccgtcctcggtgtcagcgaacccgggagaaaccgtcaagatcacctgctctggggatgacagctatggttatggctggttccagcagaagtctcctggcagtgcccctgtcactgtggtctatcacaacaacaacagaccctcggacatcccttcacgattctccggttccaaatccggctccacagccacattaaccatcactggggtccaagtcgaggacgaggctgtctattactgtgggagctacgacagcactactccttttggggccgggacaaccctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 31 - DNA, 648 bp, Gallus gallus
gcgctgactcagccgtcctcagtgtcagcgaacccaggagaaaccgtcaagatcacctgctccgggggtggcagctatgctggaagttactattatggctggtaccagcagaaggcacctggcagtgcccctgtcactctgatctatctgaacaacaacagaccctcaaacatcccttcgcgattctccggttccaaatccggctccacagccacattaaccatcactggggtccaagccgacgacgaggctgtctatttctgtgggagcatagacagcagcaacgttggtatatttggggccgggacaaccctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 32 - DNA, 636 bp, Gallus gallus
gcgctgactcagccgtcctcggtgtcagcgaacccaggagaaaccgtcaagatcacctgccctgggggcaactataattatggttggttccaacagaagtctcctggcagtgcccctgtcactgtgatctataatggcaataacagaccctcgaacatcccttcacgattctccggttccacatccggctccacaaacacattaaccatcactggggtccaagccgacgacgaggctgtctatttctgtgggagcttcgacagcagcggtactgatgctatatttggggccgggacaaccctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa